(19) United States
(12) Patent Application Publication
(10) Pub. No.: US 2025/0353903 A1
(43) Pub. Date: Nov. 20, 2025

(54) Bovine Preputial Epithelium Produces Antibodies Against Tritrichomonas foetus following Aerosol Application of Synthetic mRNA

(71) Applicants: Amelia R. Woolums, Starkville, MS (US); Philip J Santangelo, Atlanta, GA (US); Daryll Vanover, Atlanta, GA (US); Elmer Heath King, Starkville, MS (US); Merrilee Thoresen, Starkville, MS (US)

(72) Inventors: Amelia R. Woolums, Starkville, MS (US); Philip J Santangelo, Atlanta, GA (US); Daryll Vanover, Atlanta, GA (US); Elmer Heath King, Starkville, MS (US); Merrilee Thoresen, Starkville, MS (US)

(21) Appl. No.: 19/214,001

(22) Filed: May 20, 2025

Related U.S. Application Data
(60) Provisional application No. 63/649,931, filed on May 20, 2024.

(51) Int. Cl. C07K 16/20; A61K 9/12; A61K 39/00; A61K 48/00; A61P 33/02

(52) U.S. Cl. CPC C07K 16/20; A61K 9/12; A61K 48/005; A61K 48/0075; A61P 33/02; A61K 2039/505; A61K 2039/545; C07K 2319/02

(57) ABSTRACT

A synthetic mRNA construct encoding for an antibody against a cell surface antigen of Tritichomonas foetus (Tf), said synthetic mRNA construct comprising a peptide sequence selected from the group consisting of SEQ ID NO: 7, SEQ ID NO: 13, SEQ ID NO: 17, SEQ ID NO: 23, SEQ ID NO: 26, SEQ ID NO: 28, SEQ ID NO: 31, SEQ ID NO: 35, SEQ ID NO: 37, SEQ ID NO: 39, SEQ ID NO: 41, and SEQ ID NO: 44 for administration to a mammal to induce expression of the antibody. Also disclosed is a method for administering the synthetic mRNA construct to the preputial tissue of a male bovine to treat Tritichomonas foetus.

### CROSS-REFERENCE TO RELATED APPLICATION

This application claims the benefit of U.S. provisional application No. 63/649,931, filed on May 20, 2024, the disclosure of which is hereby incorporated by reference in its entirety as if fully set forth herein.

### STATEMENT OF GOVERNMENT SUPPORT

This invention was made with government support under grant no. 2021-67016-34571 awarded by the USDA National Institute of Food and Agriculture and grant no. 401240 awarded by the USDA Section 1433 Capacity Building Grant. The Government has certain rights in the invention.

### INCORPORATION OF MATERIAL OF XML SEQUENCE LISTING BY REFERENCE

The sequence listing submitted herewith as an XML file named “SequenceListing” created on May 20, 2024, which is 82 kilobytes in size, is hereby incorporated by reference in its entirety.

### BACKGROUND OF THE INVENTION

Bulls and cows are susceptible to the protozoal parasite, Tritrichomonas foetus, which prevents cows from delivering healthy calves. When bulls are infected with T. foetus, they typically remain infected for life, and since there is no legal effective treatment, infected bulls must be killed since the bulls can no longer be used for breeding. Although cows are not infected for life when exposed to this parasite, it prevents them from delivering healthy calves, and they can be reinfected by an infected bull.

Cows being unable to deliver calves leads to substantial financial loss for farmers. Accordingly, there is a need for an effective treatment for bulls to rid them of this parasite and avoid reinfection of cows during breeding.

### SUMMARY OF THE INVENTION

The present invention may be described by the following sentences:

1. In a first aspect, the present invention relates to a synthetic mRNA construct encoding for an antibody against cell surface antigen Tritichomonas foetus (Tf), said synthetic mRNA construct comprising a peptide sequence selected from the group consisting of SEQ ID NO: 7, SEQ ID NO: 13, SEQ ID NO: 17, SEQ ID NO: 23, SEQ ID NO: 26, SEQ ID NO: 28, SEQ ID NO: 31, SEQ ID NO: 35, SEQ ID NO: 37, SEQ ID NO: 39, SEQ ID NO: 41, and SEQ ID NO: 44 for administration to a mammal to induce expression of the antibody.
2. The synthetic mRNA construct of sentence 1, further comprising a secretion signal peptide.
3. The synthetic mRNA construct of any one of sentences 1-2, further comprising a membrane anchor signal peptide.
4. The synthetic mRNA construct of sentence 3, wherein the membrane anchor signal peptide has a peptide sequence selected from the group consisting of SEQ ID NO: 12, SEQ ID NO: 22, and SEQ ID NO: 30.
5. The synthetic mRNA construct of any one of sentences 1-4, wherein the mammal is selected from the group consisting of a bovine and a cat.
6. The synthetic mRNA construct of any one of sentences 1-5, wherein the mammal is a male bovine.
7. In a second aspect, the present invention relates to a method for treating Tritichomonas foetus in a male bovine, including a step of administering the synthetic mRNA construct of any one of sentences 1-4 to a preputial tissue of a male bovine.
8. The method of sentence 7, wherein the synthetic mRNA is administered to a preputial fornix of the male bovine.
9. The method of any one of sentences 7-8, wherein a dose of 0.5 mg to 10 mg, or from about 0.75 mg to about 7.5 mg, or from about 1.0 mg to about 5 mg, or from about 1.5 mg to about 3 mg of the mRNA construct is administered.
10. The method of any one of sentences 7-8, wherein a dose of 1.0 mg to about 5 mg of the mRNA construct is administered.
11. The method of any one of sentences 7-10, wherein the mRNA construct is administered via an aerosol.
12. The method of any one of sentences 7-11, wherein the mRNA construct is administered via an atomization device.
13. The method of any one of sentence 7-12, wherein the mRNA construct is suspended in a carrier.
14. The method of sentence 13, wherein the carrier is selected from the group consisting of molecular grade water, lipid nanoparticles, liposomes, poly-based nanoparticles, cell-penetrating peptides, extracellular vesicles, and combinations thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows In Vivo Imagining System (IVIS) images of preputial skin samples from calves treated with a high dose (3 mg) of anti-Tf 1.17 synthetic mRNA, left (anchored), middle (secreted) and right negative control. Radiance values were normalized to background observed on the negative control tissue.

FIG. 2 shows average radiance determined via IVIS for preputial skin samples from calves treated with a high dose (3 mg) of either an anchored or secreted version of anti-Tf 1.17 synthetic mRNA (3/treatment group) and a negative control calf. (mean±SD)

FIG. 3A shows a chart of chemiluminescence signal relative to molecular mass of skin lysates (kDa), where the solid red lines and dashed red lines indicate low dose, the solid blue lines and dashed blue lines indicate high dose, and the solid green and dashed green lines indicate a negative control calf. The solid lines indicate secreted versions and the dashed lines indicate anchored versions.

FIG. 3B shows a capillary-based western blot (JESS) image showing the detection of NanoLuc labelled secreted or anchored anti-Tf 1.17 via capillary-based western blot in preputial skin lysates from calves treated with a high (3 mg) or low (1.5 mg) dose of synthetic mRNA.

FIG. 4 shows Hematoxylin and Eosin (H&E) staining of a bull preputial epidermis. The scale bar represents 100 μm.

FIG. 5 shows the luminescence of preputial epithelial cells from bulls treated with one of 4 treatments including mRNA encoding an antibody directed against T. foetus, or negative control (NC). High luminescence of cells from cattle treated with mRNA for TF1.15 delivered in water (circles) or TF1.17 delivered in water (upright triangles) indicates expression of antibody by the bovine preputial epithelial cells treated with mRNA.

FIG. 6 shows the luminescence of preputial secretions from bulls treated with one of 4 treatments including mRNA encoding an antibody directed against T. foetus, or negative control (NC). High luminescence of secretions from cattle treated with mRNA for TF1.15 (circles) or TF1.17 (upright triangles) indicates expression of antibody by mRNA treated cells that was released into the secretions.

FIG. 7 shows Immunofluorescence detection of NanoLuciferase reporter molecule linked to an anti TF1.17 antibody in preputial epithelial cells of a bull treated with mRNA in water 14 days before (left). Green fluorescence of multiple layers of epithelial cells is evident (white arrow). In contrast, no green fluorescence is seen in the same region of preputial tissue of a bull treated with water alone (right).

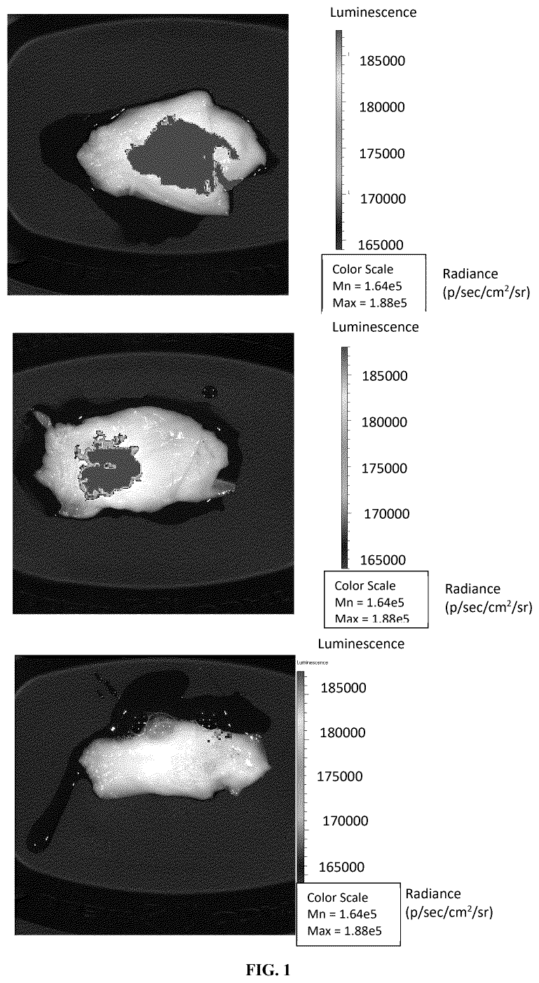

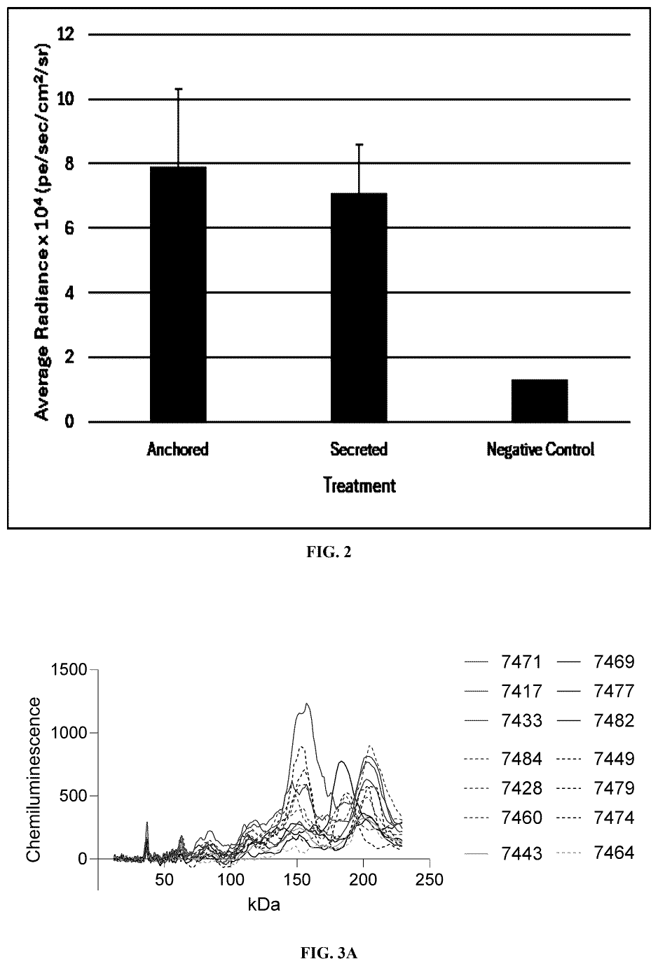

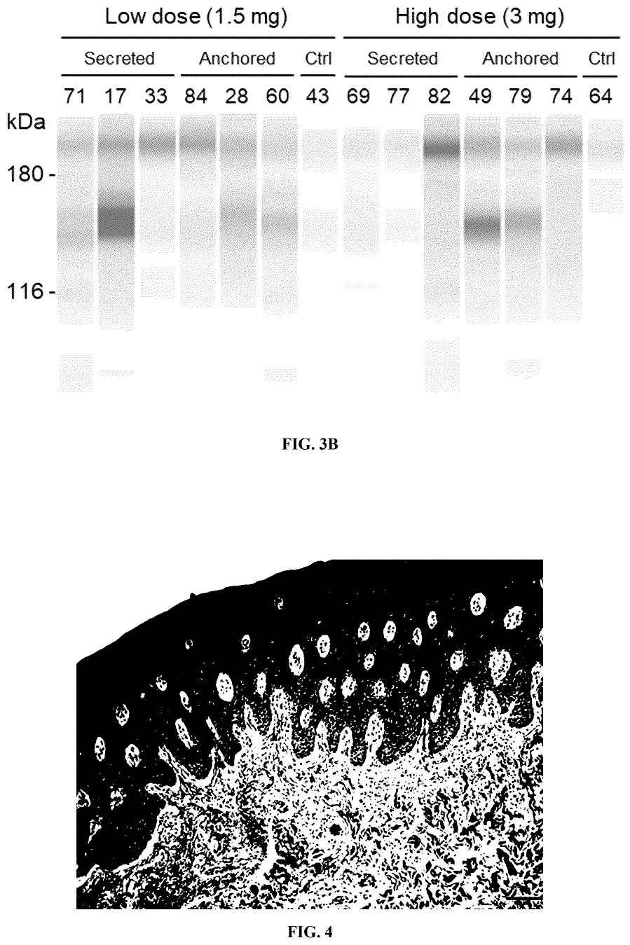

### DETAILED DESCRIPTION OF THE INVENTION

### Definitions

As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g. in light of their history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

The present invention relates to a synthetic mRNA construct encoding for an antibody against cell surface antigen Tritichomonas foetus (Tf) having any one of the following peptide sequences SEQ ID NO: 7, SEQ ID NO: 13, SEQ ID NO: 17, SEQ ID NO: 23, SEQ ID NO: 26, SEQ ID NO: 28, SEQ ID NO: 31, SEQ ID NO: 35, SEQ ID NO: 37, SEQ ID NO: 39, SEQ ID NO: 41, and SEQ ID NO: 44 for administration to a mammal to induce expression of the antibody. In another aspect, the present invention relates to methods for treating Tritichomonas foetus by administering the synthetic mRNA construct to a preputial tissue of a male bovine.

In some embodiments the synthetic mRNA is a secreted or membrane anchored version. When the synthetic mRNA is a secreted version, it comprises a peptide sequence selected from the group consisting of SEQ ID NO: 7, SEQ ID NO: 13, SEQ ID NO: 17, SEQ ID NO: 23, SEQ ID NO: 26, SEQ ID NO: 28, SEQ ID NO: 31, SEQ ID NO: 35, SEQ ID NO: 37, SEQ ID NO: 39, SEQ ID NO: 41, and SEQ ID NO: 44.

Suitable secretion signal peptides are known in the art or can be predicted using standard software known to skilled persons. Any suitable known or predicted secretion signal peptide can be employed in the present invention.

When the synthetic mRNA is a membrane anchored version, it comprises a membrane anchor signal peptide sequence selected form the group consisting of SEQ ID NO: 12, SEQ ID NO: 22, and SEQ ID NO: 30.

The synthetic mRNA construct of the present invention may be suitable to treat a mammal, such as a bovine and a cat. In some embodiments the mammal is a male bovine.

The present invention also relates methods for treating Tritichomonas foetus in a male bovine, including a step of administering the synthetic mRNA construct disclosed herein to a preputial tissue of a male bovine. Typically, the synthetic mRNA is administered to a preputial fornix of the male bovine. Suitable doses may be selected from 0.5 mg to 10 mg, or from about 0.75 mg to about 7.5 mg, or from about 1.0 mg to about 5 mg, or from about 1.5 mg to about 3 mg of the mRNA construct. Preferably, the mRNA construct is administered at a dose of 1.0 mg to about 5 mg. The mRNA construct may be administered via aerosol, for example, using an atomization device.

In some embodiments, the mRNA construct is suspended in a carrier. Suitable carriers may be selected from the group consisting of molecular grade water, lipid nanoparticles, liposomes, poly-based nanoparticles, cell-penetrating peptides, extracellular vesicles, and combinations thereof.

The present invention evaluated levels of Tritichomonas foetus (Tf) specific bovine IgG in preputial skin of bull calves following aerosol treatment with synthetic mRNA encoding for an antibody against the cell surface antigen Tf 1.17. The goal was to determine if there were differences in expressed levels of antibody three weeks following treatment, when using a low or high dose of synthetic mRNA for inducing expression of the antibody.

Objective 1: Transfect the preputial epithelium of bull calves with a high or low dose of synthetic mRNA encoding for an anti-Tf 1.17 antibody.

Objective 2: Detect antibody in the preputial skin of calves, 3 weeks following treatment and determine whether there was a difference in expression levels between high or low doses and the anchored or secreted versions of the synthetic mRNA.

Detection of luminescence in preputial skin samples of calves that received a high (3 mg) dose of synthetic mRNA confirmed the production of anti-TF 1.17 antibody and average radiance values were comparable for both anchored and secrete versions of the synthetic mRNA.

While luminescent signal above background was not detectable in skin samples from calves that received the low (1.5 mg) dose of synthetic mRNA, NanoLuc labelled bovine IgG was detected via western blot in skin lysates from calves treated with both the low and high doses of synthetic mRNA confirming that anti-Tf 1.17 antibody was produced in all of the treated calves.

### EXAMPLES

### Example 1

### Materials and Methods

Four treatment groups consisting of 3 bull calves each included a high (3 mg) or low (1.5 mg) dose of synthetic mRNA, encoding for expression of antibodies against Tf. Additionally, two negative control calves received no mRNA treatment. The light chain sequence included a NanoLuc reporter label to facilitate identification of the expressed protein and differentiate it from naïve bovine IgG. Synthetic mRNA was suspended in molecular grade water (carrier) and applied via a mucosal atomization device to the preputial fornix of each calf. Two negative control calves were treated with carrier only.

Three weeks following treatment, calves were euthanized, and preputial epithelium was collected and frozen. Upon thawing, the superficial epidermis was trimmed back to expose the dermis, diluted NanoGlo (Promega) substrate was applied and the tissue was imaged for luminescent signal using an in vivo imaging system (IVIS).

After correcting for background noise using values from the negative control tissue, average radiance was determined for each of the treated calf tissues. Following imaging, areas exhibiting high radiance were excised via 8 mm biopsy punches obtained from the skin samples and a protein lysate was prepared from them. Capillary-based western blots for NanoLuc labelled bovine IgG were prepared and imaged using a Jess automated system (biotechne).

Results. Skin samples from calves that were treated with the high dose (3 mg) of the secreted or anchored mRNA constructs had areas of radiance that ranged from 1.8-2.4×105, while low dose calves exhibited no radiance following background correction when imaged with IVIS. In contrast, NanoLuc labelled bovine IgG was detected in western blots of skin lysates from all of the calves that were treated with synthetic mRNA. Chemiluminescent signals were detected, and the bands were consistent with products the size of bovine IgG with NanoLuc labelled light chains.

Conclusions. Detection of NanoLuc in the skin samples from treated calves, via both IVIS imaging and western blot, confirmed that aerosol application of synthetic mRNA to preputial skin induced the production of bovine IgG against the Tf 1.17 cell surface antigen. Using synthetic mRNA to produce antibodies against Tf at the site of infection, in combination with systemic vaccination, may provide a new therapeutic approach to prevent or clear Tf infection in bulls.

### Example 2

Twelve bulls were treated with synthetic mRNA encoding one of two different antibodies directed against T. foetus (TF1.17 or TF1.15), engineered to remain anchored to expressing cells (ANC) and linked to a NanoLuciferase reporter molecule which allowed detection of expressed antibody by a rapid luminescence assay. Each synthetic mRNA was administered in either water alone (W) or water and a polymer delivery agent (P). The treated groups (n=3 bulls per group) were:
TF1.15 ANC W
TF1.15 ANC P
TF1.17 ANC W
TF1.17 ANC P

Two bulls were treated with water alone (negative control group). Treatments were applied by spray to the preputial fornix of each animal. Preputial cells and secretions were collected from treated bulls before treatment (day 0) and on days 1, 4, 8, 11 and 14 after treatment. Cells and secretions were tested by luminescence assay for the presence of NanoLuciferase, which is not naturally produced by cattle and therefore represents the expression of the synthetic mRNA-encoded antibody.

On day 1, high luminescence values were measured in preputial cells collected from all bulls treated with the synthetic mRNA for TF1.15 or TF1.17 when administered in water (FIG. 4). One bull in the TF1.17 group also demonstrated increased luminescence on day 4.

Secretions from the preputial cavity were also tested by luminescence assay and, similar to cells, increased luminescence indicating antibody expression was demonstrated in secretions from all bulls treated with the synthetic mRNA for either antibody, anti-TF1.15 or anti-TF1.17, in water, on day 1. Antibody expression indicated by a luminescence increase over negative controls was seen in one animal per group on days 4, 8, and 11 (FIG. 5):

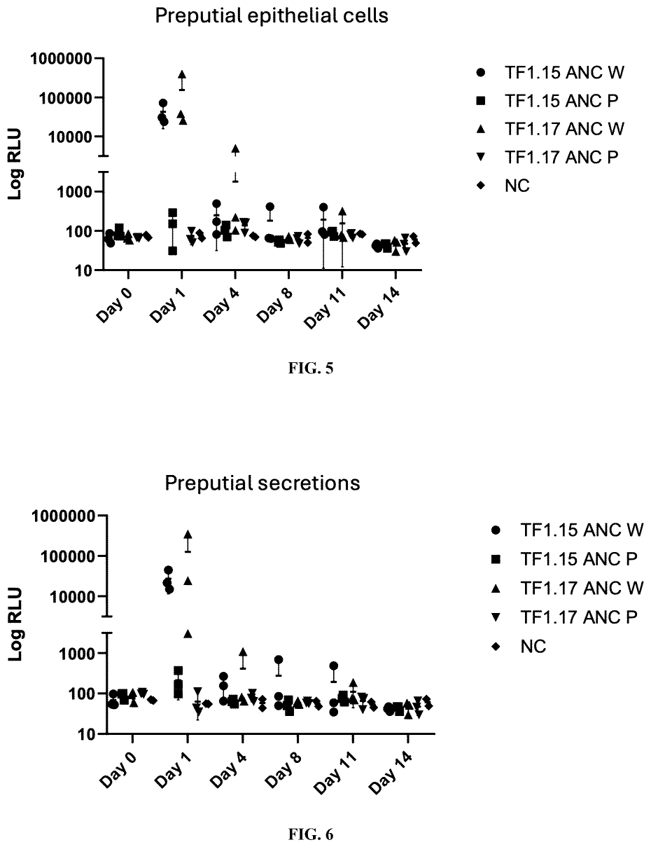

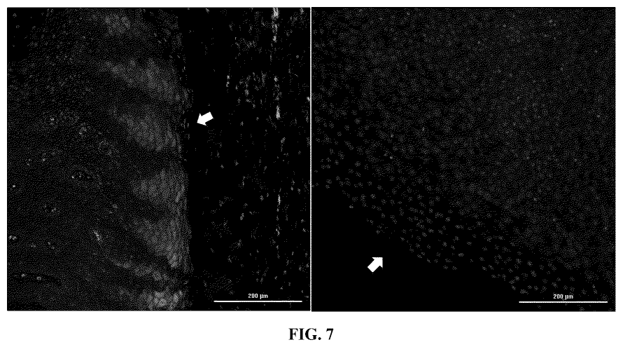

Preputial tissues from treated bulls collected after the animals were euthanized were tested for antibody expression by immunofluorescence assay to detect NanoLuciferase. Fluorescence indicates expression of antibody fused to NanoLuciferase, which is not naturally produced by cattle. Green fluorescence in preputial epithelial cells of tissue from a bull treated with mRNA for TF1.17 delivered in water 14 days previously can be seen in FIG. 6. No fluorescence is seen in tissue from the negative control animal treated only with water.

These data confirm that bulls treated with mRNA encoding either one of two antibodies, TF1.15 or TF1.17, that can bind to T. foetus, were expressed by preputial epithelial cells and were found in the secretions of the preputial tissue, where T. foetus infection occurs.

### Single Chain Antibodies

### 1. Single Chain TF1.17—Secreted

SEQ ID NO: 7—Single chain TF1.17 secreted NanoLuc-RNA620-JYJ069 SC-Tf1.17 sIgG w/ Nluc

5′UTR
SEQ ID NO: 1
AAGCTTTAATACGACTCACTATAGGGAAATAAGAGAGAAAAGAAGAGTAAGAAGAAATATAAGA
GCCACC
ORF
SEQ ID NO: 2
ATGAAATGGGTCACTTTTATCAGCCTCTTGTTTCTGTTCAGCAGTGCCTATTCTCAAGC
CGTGGTTACGCAGGAAAGTGCCCTGACAACCTCACCTGGGGAAACAGTAACTCTGA
CCTGCAGGAGCTCAACTGGAGCAGTTACAACTTCCAACTACGCCAACTGGGTGCAGG
AGAAGCCCGACCATCTGTTTACAGGCCTGATTGGCGGCACCAACAACCGGGCCCCTG
GTGTACCCGCTCGCTTTTCAGGTAGCCTTATAGGAGATAAAGCTGCGCTAACGATCAC
TGGTGCACAGACAGAGGATGAAGCCATCTATTTCTGCGCGTTGTGGTACTCAAATCTG
TGGGTTTTCGGCGGCGGCACCAAGCTAACTGTGCTGCGCTCTGATGCTGAGCCTAGC
GTTTTCCTGTTTAAACCAAGCGACGAACAGCTCAAGACTGGGACAGTATCTGTTGTG
TGTCTTGTGAACGACTTTTATCCTAAGGACATCAATGTGAAGTGGAAGGTGGATGGCG
TGACTCAGTCATCTTCTAACTTCCAGAATAGCTTCACAGATCAGGATAGCAAGAAAAG
CACATATAGCCTTAGTTCTATCCTTACGCTACCCTCCAGTGAGTACCAGTCCCATGATG
CCTACACATGTGAAGTTAGCCACAAGTCTCTTACAACAACCCTGGTAAAGAGCTTCA
GTAAAAATGAATGTGGCGGTGGAGGATCTGGAGGGGGCGGGTCCGGGGGTGGGGGA
TCCGGAGGAGGCGGGTCAGGTGGTGGTGGCAGCGGAGGCGGCGGATCTGGTGGCGG
CGGCTCCGGCGGGGGTGGCTCCGGGGGAGGAGGCTCGGGCGGTGGTGGGAGTGGA
GGAGGTGGCTCCGGCGGCGGCGGTTCCCAAGTCCAGTTAAAGCAGGCAGGCCCAGG
GCTCGTTCAACCGAGTCAATCTCTGTCTATAACGTGCACCGTCAGCGGATTTAGTCTC
TCCAATTATGGAGTCCACTGGGTGCGCCAGTCCCCGGGAAAGGGGCTGGAATGGCTG
GGCTTAATCTGGGGCGGGGGGCGAAGTGATTATAATGCAGCCTTTATTTCCCGACTCA
CCATTAGCAAGGATAACTCTAAGAGCCAGGTCTTCTTCACCATGAATTCCCTGCAGGT
CAATGACACGGGAATCTACTACTGTGTCCGCAATGGTGGCGACCCCGCTTGGTTCGCT
TACTGGGGCCAGGGCACGCTCGTAACCGTGTCCGCAGCTAGCACAAGCGCCCCCAAA
GTATATCCTTTGACCTCTTGCTGCGGGGACAAGTCAAGCTCAAGAGTGACATTGGGCT
GCCTGGTCTCAAGTTATATGCCTGAGCCGGTGACTGTTACCTGGAATAGTGGTGCGCT
GAAATCTGGGGTGCACACCTTCCCTGCGGTGCTCCAGAGCAGCGGCCTCTACTCTCT
CTCCTCCATGGTTACCGTCCCAGGAAGTACCTCCGGGCAGACGTTTACCTGCAACGTG
GCCCACCCTGCAAGCAGTACAAAAGTCGACAAGGCTGTGGGCGTTTCTTCAGACTGT
AGTAAGCCTAACAATCAGCATTGTGTGAGGGAGCCATCGGTGTTCATCTTTCCCCCGA
AGCCAAAAGACACTCTAATGATTACTGGTACACCTGAAGTCACCTGTGTCGTAGTCA
ACGTTGGCCATGACAATCCAGAGGTCCAGTTTTCGTGGTTTGTGGACGACGTCGAGG
TGCATACAGCCAGAACCAAACCCCGGGAGGAGCAGTTCAACTCAACCTACCGGGTC
GTCTCGGCCCTCCCTATCCAGCACCAGGACTGGACGGGGGGCAAGGAGTTTAAGTGC
AAGGTTAACATCAAAGGCTTGTCAGCGAGTATAGTGCGGATCATATCCAGATCAAAAG
GACCAGCTCGAGAGCCCCAGGTGTATGTTTTGGACCCACCAAAGGAAGAGCTGAGC
AAAAGCACAGTGTCTGTCACATGCATGGTGATCGGCTTCTACCCCGAAGATGTGGAC
GTGGAGTGGCAGAGAGACCGGCAGACCGAGAGCGAAGACAAGTACAGGACCACCC
CGCCACAGCTGGATGCAGATCGTTCCTACTTCCTGTACAGCAAGCTGCGCGTGGACA
GGAACTCCTGGCAGCGGGGGGGAACTTACACTTGCGTGGTCATGCACGAAGCCCTCC
ACAACCACTACATGCAGAAGTCTACCTCTAAAAGCGCCGGAAAAGGGGGCGGAGGC
TCGGTATTCACCTTGGAAGATTTCGTGGGTGATTGGAGGCAAACCGCTGGCTACAAC
CTGGACCAAGTGCTGGAGCAAGGGGGCGTTTCAAGTCTTTTTCAAAACCTCGGTGTG
TCCGTGACACCCATTCAGAGGATCGTACTGTCGGGGGAGAATGGGCTGAAGATAGAT
ATTCATGTGATAATCCCCTATGAAGGACTCAGTGGGGACCAGATGGGCCAAATTGAAA
AAATCTTCAAAGTGGTATATCCAGTTGATGACCACCACTTCAAGGTGATTCTGCACTAT
GGGACCCTGGTTATTGACGGTGTCACGCCCAACATGATCGACTACTTTGGGCGGCCCT
ACGAGGGGATTGCCGTGTTTGATGGAAAGAAAATAACAGTGACCGGGACTTTATGGA
ATGGCAACAAAATTATTGATGAGCGACTGATCAACCCTGATGGATCTCTACTTTTCCGT
GTAACTATTAATGGAGTGACTGGCTGGAGACTGTGTGAGAGAATCCTTGCATGATAG
GS-linker (contained within ORF)
SEQ ID NO: 3
GGGGGCGGAGGCTCG
Nluc (contained within ORF)
SEQ ID NO: 4
GTATTCACCTTGGAAGATTTCGTGGGTGATTGGAGGCAAACCGCTGGCTACAACCTG
GACCAAGTGCTGGAGCAAGGGGGCGTTTCAAGTCTTTTTCAAAACCTCGGTGTGTCC
GTGACACCCATTCAGAGGATCGTACTGTCGGGGGAGAATGGGCTGAAGATAGATATTC
ATGTGATAATCCCCTATGAAGGACTCAGTGGGGACCAGATGGGCCAAATTGAAAAAA
TCTTCAAAGTGGTATATCCAGTTGATGACCACCACTTCAAGGTGATTCTGCACTATGG
GACCCTGGTTATTGACGGTGTCACGCCCAACATGATCGACTACTTTGGGCGGCCCTAC
GAGGGGATTGCCGTGTTTGATGGAAAGAAAATAACAGTGACCGGGACTTTATGGAAT
GGCAACAAAATTATTGATGAGCGACTGATCAACCCTGATGGATCTCTACTTTTCCGTGT
AACTATTAATGGAGTGACTGGCTGGAGACTGTGTGAGAGAATCCTTGCA
3′ UTR
SEQ ID NO: 5
GCTCGCTTTCTTGCTGTCCAATTTCTATTAAAGGTTCCTTTGTTCCCTAAGTCCAACTACTAAA
CTGGGGGATATTATGAAGGGCCTTGAGCATCTGGATTCTGCCTAATAAAAAACATTTA
TTTTCATTGC
Not-I
SEQ ID NO: 6
GCGGCCGC

### 2. Single Chain TF1.17—Anchored

SEQ ID NO: 13—Single chain TF1.17 anchored NanoLuc-RNA621-JYJ070 SC-Tf1.17 aIgG w/ Nluc

5′UTR
SEQ ID NO: 1
AAGCTTTAATACGACTCACTATAGGGAAATAAGAGAGAAAAGAAGAGTAAGAAGAAATATAAG
AGCCACC
ORF
SEQ ID NO: 8
ATGAAGTGGGTTACCTTCATCTCTTTGCTCTTCCTGTTTTCGAGTGCTTATTCTCAAGCTGTG
GTCACCCAGGAATCAGCTCTCACAACATCACCGGGAGAGACCGTAACTCTAACATGTA
GGAGCTCCACGGGTGCAGTTACCACAAGCAATTATGCCAACTGGGTCCAGGA
GAAGCCCGACCATCTGTTCACAGGGTTGATTGGCGGCACCAACAACCGAGCTCCCGG
GGTGCCCGCACGCTTCTCAGGGTCACTCATTGGTGATAAAGCAGCATTGACCATCACG
GGAGCCCAGACGGAAGACGAGGCCATCTACTTCTGTGCCCTGTGGTACAGCAACCTC
TGGGTGTTTGGTGGTGGTACCAAGCTGACTGTACTGAGAAGCGACGCTGAGCCATCT
GTTTTCCTCTTCAAACCCTCCGATGAGCAACTGAAAACTGGCACTGTCTCAGTAGTGT
GCCTGGTGAATGACTTCTACCCGAAGGATATCAATGTCAAATGGAAGGTTGATGGTGT
GACACAGTCCTCTTCCAATTTCCAGAACAGTTTTACCGACCAGGACAGCAAGAAGTC
CACCTATTCGCTAAGCAGCATACTGACGCTGCCTAGTTCCGAGTACCAGAGTCATGAT
GCCTACACATGTGAGGTCAGCCACAAGTCTTTGACGACCACTCTCGTGAAGAGTTTC
AGCAAAAATGAATGTGGAGGTGGCGGCAGTGGGGGGGGAGGCTCTGGAGGGGGCG
GCTCCGGCGGGGGAGGCAGTGGAGGGGGGGGCTCCGGAGGTGGGGGTTCTGGCGG
CGGTGGCTCAGGGGGGGGAGGATCTGGTGGAGGGGGATCTGGAGGGGGTGGATCAG
GTGGTGGCGGCTCGGGAGGCGGGGGTAGTCAAGTGCAGCTGAAGCAGGCCGGCCCC
GGCCTGGTACAGCCTAGCCAGTCTCTTAGCATCACGTGCACAGTGAGCGGTTTCTCGT
TGTCCAACTATGGAGTGCACTGGGTTAGGCAAAGTCCAGGCAAGGGCCTGGAGTGGT
TGGGGCTTATTTGGGGGGGAGGACGTTCGGATTACAATGCTGCCTTCATATCACGCCT
CACCATTTCGAAGGACAATTCTAAATCTCAGGTGTTCTTCACTATGAACTCCCTCCAG
GTCAATGACACTGGTATTTATTACTGCGTCAGGAACGGAGGAGACCCCGCCTGGTTTG
CGTACTGGGGCCAAGGAACCTTAGTGACAGTTAGTGCGGCCTCAACAAGCGCCCCGA
AAGTTTACCCTCTGACTAGCTGCTGTGGCGACAAGAGCTCCTCACGAGTCACTCTGG
GCTGCCTTGTCAGTTCCTACATGCCAGAGCCTGTGACAGTCACCTGGAACTCTGGGG
CGTTGAAAAGCGGAGTCCACACGTTTCCAGCAGTGCTGCAGTCCTCCGGTCTTTATTC
TCTGAGCAGCATGGTCACTGTGCCCGGAAGCACTTCGGGGCAGACCTTTACTTGTAA
TGTGGCCCACCCTGCCTCCTCAACAAAAGTGGACAAGGCCGTCGGCGTATCCAGTGA
CTGCAGCAAGCCAAATAACCAGCATTGTGTAAGAGAACCGAGCGTATTCATATTTCCC
CCTAAACCAAAAGACACCCTGATGATTACTGGGACACCAGAAGTCACCTGCGTGGTG
GTGAACGTCGGGCATGACAACCCCGAAGTTCAGTTCTCCTGGTTCGTGGACGATGTG
GAGGTGCATACCGCGCGGACCAAGCCAAGAGAGGAACAGTTTAATAGTACGTACAGG
GTTGTCTCCGCCCTGCCCATCCAGCACCAGGATTGGACCGGGGGGAAAGAGTTTAAG
TGCAAGGTTAATATTAAAGGTCTCTCAGCTTCCATTGTGCGCATCATCAGCAGATCCA
AGGGGCCGGCCCGGGAGCCCCAGGTGTATGTGCTTGACCCACCCAAGGAAGAGCTG
AGCAAATCAACGGTGTCTGTTACATGCATGGTGATCGGCTTCTATCCTGAGGATGTGG
ACGTGGAATGGCAGAGGGACAGACAAACGGAGTCTGAAGATAAGTACCGCACCACC
CCTCCTCAGTTGGATGCTGATAGGTCCTATTTTCTCTACTCTAAGCTCCGCGTGGATAG
AAACAGCTGGCAGCGGGGCGGCACTTACACCTGTGTGGTCATGCATGAAGCACTACA
CAACCACTACATGCAGAAGTCTACGTCCAAAAGTGCAGGAAAAGGAGGCGGTGGGT
CAGTTTTTACTCTGGAGGATTTTGTGGGAGACTGGCGCCAAACAGCTGGCTACAACC
TGGACCAAGTATTAGAACAAGGAGGTGTGAGCTCCCTCTTTCAGAATCTGGGGGTGA
GCGTAACACCTATTCAAAGAATCGTCCTGTCAGGGGAGAATGGACTAAAGATTGACA
TCCATGTTATCATTCCATATGAGGGCTTATCCGGGGATCAGATGGGCCAGATCGAGAA
GATATTTAAAGTGGTGTACCCAGTTGATGACCACCACTTCAAGGTGATCCTGCACTAT
GGGACCCTCGTGATAGACGGCGTCACACCGAACATGATCGACTACTTTGGGCGGCCC
TATGAAGGCATTGCAGTCTTTGATGGAAAAAAGATCACCGTGACTGGCACTTTATGGA
ATGGAAATAAAATTATAGATGAACGGCTGATCAATCCTGATGGATCGCTACTTTTCCGT
GTGACCATAAACGGCGTCACAGGCTGGAGGCTGTGTGAACGAATCCTTGCGGGCGGT
GGTGGAAGCCCCAACAAAGGGAGCGGCACAACTTCTGGGACCACTCGGCTCTTGAG
TGGCCACACTTGCTTCACCCTTACCGGGCTCTTAGGAACGCTGGTCACCATGGGACT
GCTGACGTGATAG
GS-linker (contained within ORF)
SEQ ID NO: 9
GGAGGCGGTGGGTCA
Nluc (contained within ORF)
SEQ ID NO: 10
GTTTTTACTCTGGAGGATTTTGTGGGAGACTGGCGCCAAACAGCTGGCTACAACCTG
GACCAAGTATTAGAACAAGGAGGTGTGAGCTCCCTCTTTCAGAATCTGGGGGTGAGC
GTAACACCTATTCAAAGAATCGTCCTGTCAGGGGAGAATGGACTAAAGATTGACATCC
ATGTTATCATTCCATATGAGGGCTTATCCGGGGATCAGATGGGCCAGATCGAGAAGATA
TTTAAAGTGGTGTACCCAGTTGATGACCACCACTTCAAGGTGATCCTGCACTATGGGA
CCCTCGTGATAGACGGCGTCACACCGAACATGATCGACTACTTTGGGCGGCCCTATGA
AGGCATTGCAGTCTTTGATGGAAAAAAGATCACCGTGACTGGCACTTTATGGAATGG
AAATAAAATTATAGATGAACGGCTGATCAATCCTGATGGATCGCTACTTTTCCGTGTGA
CCATAAACGGCGTCACAGGCTGGAGGCTGTGTGAACGAATCCTTGCG
GS-linker (contained within ORF)
SEQ ID NO: 11
GGCGGTGGTGGAAGC
ANCHOR SEQUENCE (Contained within ORF)
SEQ ID NO: 12
CCCAACAAAGGGAGCGGCACAACTTCTGGGACCACTCGGCTCTTGAGTGGCCACAC
TTGCTTCACCCTTACCGGGCTCTTAGGAACGCTGGTCACCATGGGACTGCTGACG
3′UTR
SEQ ID NO: 5
GCTCGCTTTCTTGCTGTCCAATTTCTATTAAAGGTTCCTTTGTTCCCTAAGTCCAACTA
CTAAACTGGGGGATATTATGAAGGGCCTTGAGCATCTGGATTCTGCCTAATAAAAAAC
ATTTATTTTCATTGC
Not-I
SEQ ID NO: 6
GCGGCCGC

### 3. Single Chain TF1.15—Secreted

SEQ ID NO: 17—Single chain TF1.15 secreted NanoLuc-RNA622-JYJ071 SC-Tf1.15 sIgG w/Nluc

5′UTR
SEQ ID NO: 1
AAGCTTTAATACGACTCACTATAGGGAAATAAGAGAGAAAAGAAGAGTAAGAAGAAA
TATAAGAGCCACC
ORF
SEQ ID NO: 14
ATGAAGTGGGTGACCTTTATCTCGTTACTCTTCCTATTTTCTTCGGCCTACTCAGACATCGTG
ATGTCTCAGTCTCCGAGTTCCCTGGCAGTTAGCGTCGGAGAAAAAGTGACCATGAG
CTGTAAGAGTTCTCAGTCACTCCTGTACAGTAGTAATCAAAAAAACTACCTGGC
CTGGTACCAGCAGAAGCCGGGACAGAGCCCCAAACTTTTAATATACTGGGCCAGCAC
TCGCGAGTCCGGTGTCCCGGATCGCTTCACCGGATCAGGATCCGGTACAGATTTTACC
TTAACTATCTCCTCCGTGAAGGCAGAGGATTTAGCAGTCTATTACTGCCAGCAGTACTA
TAATTATCCATTCACGTTTGGAGGAGGTACCAACCTGGAGATCAAGAGGAGTGACGC
CGAGCCAAGCGTCTTTCTGTTTAAACCTTCAGATGAGCAACTAAAGACCGGGACGGT
ATCAGTGGTGTGCCTGGTGAATGACTTCTATCCTAAAGACATCAATGTGAAATGGAAG
GTTGACGGGGTCACTCAGTCCAGCTCAAATTTTCAGAATTCATTCACTGACCAGGACT
CAAAGAAGAGTACTTATTCCTTGAGTTCCATCCTTACACTTCCCTCTTCGGAATATCAG
AGCCATGACGCGTATACCTGTGAGGTGAGCCACAAATCTCTTACAACGACTTTGGTGA
AAAGCTTTTCAAAGAATGAATGTGGCGGTGGCGGTTCAGGGGGGGGCGGCTCTGGC
GGCGGTGGCAGTGGCGGCGGAGGTTCCGGGGGAGGCGGATCCGGAGGAGGGGGCA
GCGGTGGCGGCGGAAGTGGTGGCGGGGGCTCCGGGGGAGGAGGATCAGGGGGGGG
TGGGAGTGGTGGCGGGGGCTCAGGGGGAGGAGGTTCCCAAGTTCAGCTGAAGCAGA
GTGGACCCGGACTCGTACAGCCCAGCCAAAACTTGTCCATTACATGTACAGTCTCTGG
CTTCAGCCTGACGACCTATGGCGTGCACTGGGTCAGGCAAAGTCCTGGCAAGGGCCT
GGACTGGCTCGGGGTCATTTGGGGGGGGGGCTCCACAGACTTCAATGCAGCCTTTAT
GTCGAGGCTCTCCATCACTAAAGATAACAGCAAATCCCAGGTCTTCTTCAAGATGAAC
TCCCTGCAGGCCGATGATACAGCCATCTACTACTGTGCCAAGAGCCTTCTATCAAGCA
TGGATTACTGGGGCCAAGGCACGTCCGTCACCGTCAGTTCAGCGTCTACGTCGGCTC
CTAAAGTCTATCCGCTCACTTCGTGCTGCGGGGACAAATCGTCATCTAGGGTTACACT
GGGCTGCCTAGTTTCTAGCTACATGCCCGAACCCGTTACAGTTACGTGGAATAGCGGG
GCTCTTAAGTCTGGTGTGCACACCTTCCCCGCAGTGTTGCAGTCATCCGGTCTCTACT
CCCTGAGCTCCATGGTGACGGTGCCCGGGAGCACCAGCGGCCAAACATTTACTTGCA
ATGTAGCGCATCCAGCATCGTCGACAAAAGTAGACAAAGCTGTGGGAGTTAGCTCGG
ACTGCAGCAAGCCCAACAACCAGCACTGCGTAAGAGAGCCCTCTGTCTTTATCTTCC
CACCAAAACCAAAGGACACCCTCATGATTACCGGGACTCCCGAAGTAACCTGTGTAG
TTGTGAATGTTGGCCACGACAATCCAGAGGTGCAGTTCTCCTGGTTCGTGGATGACGT
GGAAGTTCATACTGCTCGCACCAAGCCTCGGGAGGAACAGTTTAACAGTACCTACAG
AGTCGTGTCAGCCCTGCCTATCCAGCATCAGGATTGGACAGGGGGGAAAGAGTTCAA
GTGCAAAGTGAACATCAAAGGGCTCAGCGCCAGCATTGTCCGTATAATTTCCAGGTCT
AAGGGCCCTGCTCGAGAACCCCAGGTATACGTGCTGGATCCACCCAAGGAGGAGCTC
TCCAAGTCAACCGTTTCCGTGACCTGCATGGTGATTGGATTCTACCCTGAGGACGTCG
ATGTGGAGTGGCAGAGAGACCGGCAGACGGAGAGTGAAGACAAGTACAGAACCAC
CCCACCCCAACTGGATGCTGACCGATCCTACTTCCTGTATAGCAAGCTGCGGGTGGAT
AGGAATAGCTGGCAGCGGGGAGGCACATATACATGTGTGGTCATGCATGAAGCACTT
CACAACCACTACATGCAGAAATCTACATCTAAGTCTGCTGGGAAGGGTGGCGGAGGC
AGCGTTTTTACACTGGAAGATTTTGTGGGCGACTGGAGACAGACTGCCGGCTACAAC
TTGGACCAAGTGCTCGAGCAGGGCGGGGTCAGCTCCTTGTTCCAGAACCTTGGGGTT
AGCGTGACTCCTATTCAACGAATTGTTCTGAGTGGCGAAAACGGATTAAAGATTGATA
TTCATGTGATCATCCCGTATGAGGGGCTGTCGGGGGACCAGATGGGCCAGATCGAGA
AAATCTTTAAAGTGGTGTACCCTGTCGACGATCACCACTTCAAAGTCATCCTGCACTA
TGGAACCCTGGTGATAGATGGTGTAACGCCAAACATGATAGACTATTTTGGTCGGCCT
TACGAGGGCATAGCCGTGTTTGATGGCAAAAAGATAACAGTAACTGGTACCCTGTGG
AACGGCAACAAGATTATTGACGAACGTCTGATCAACCCTGATGGATCGCTACTGTTCC
GGGTGACCATTAATGGAGTCACTGGCTGGCGCCTGTGTGAAAGAATCCTGGCTTGATAG
GS-linker (contained within ORF)
SEQ ID NO: 15
GGTGGCGGAGGCAGC
Nluc (contained within ORF)
SEQ ID NO: 16
GTTTTTACACTGGAAGATTTTGTGGGCGACTGGAGACAGACTGCCGGCTACAACTTG
GACCAAGTGCTCGAGCAGGGGGGGTCAGCTCCTTGTTCCAGAACCTTGGGGTTAGC
GTGACTCCTATTCAACGAATTGTTCTGAGTGGCGAAAACGGATTAAAGATTGATATTC
ATGTGATCATCCCGTATGAGGGGCTGTCGGGGGACCAGATGGGCCAGATCGAGAAAA
TCTTTAAAGTGGTGTACCCTGTCGACGATCACCACTTCAAAGTCATCCTGCACTATGG
AACCCTGGTGATAGATGGTGTAACGCCAAACATGATAGACTATTTTGGTCGGCCTTAC
GAGGGCATAGCCGTGTTTGATGGCAAAAAGATAACAGTAACTGGTACCCTGTGGAAC
GGCAACAAGATTATTGACGAACGTCTGATCAACCCTGATGGATCGCTACTGTTCCGGG
TGACCATTAATGGAGTCACTGGCTGGCGCCTGTGTGAAAGAATCCTGGCT
3′UTR
SEQ ID NO: 5
GCTCGCTTTCTTGCTGTCCAATTTCTATTAAAGGTTCCTTTGTTCCCTAAGTCCAACTACTAA
ACTGGGGGATATTATGAAGGGCCTTGAGCATCTGGATTCTGCCTAATAAAAAAC
ATTTATTTTCATTGC
Not-I
SEQ ID NO: 6
GCGGCCGC

4. Single chain TF1.15—Anchored

SEQ ID NO: 23—Single chain TF1.15 anchored NanoLuc RNA623-JYJ072 SC-Tf1.15 aIgG w/Nluc

5′UTR
SEQ ID NO: 1
AAGCTTTAATACGACTCACTATAGGGAAATAAGAGAGAAAAGAAGAGTAAGAAGAAATATAA
GAGCCACC
ORF
SEQ ID NO: 18
ATGAAATGGGTCACGTTCATCAGTCTGCTATTTTTGTTCTCCTCCGCTTACAGCGACATTGT
GATGAGTCAAAGTCCCTCTTCTCTGGCGGTGAGTGTCGGAGAGAAAGTGACAAT
GTCTTGCAAGAGTAGCCAGTCATTGCTGTACAGCAGTAACCAGAAGAACTATCTCGC
TTGGTACCAGCAGAAACCAGGTCAGTCACCTAAACTGCTGATATACTGGGCATCAAC
CCGAGAATCAGGCGTACCAGACCGGTTTACTGGGTCCGGGAGCGGTACGGACTTCAC
GCTTACAATATCATCCGTAAAAGCAGAGGACCTTGCAGTTTACTACTGCCAGCAGTAC
TATAATTATCCCTTCACATTCGGCGGAGGCACCAACCTGGAAATCAAGAGATCAGATG
CTGAACCATCTGTGTTTCTTTTTAAGCCCTCCGACGAACAGCTGAAGACAGGTACTGT
CAGTGTGGTATGCTTAGTTAACGACTTCTACCCGAAGGATATTAATGTCAAGTGGAAG
GTGGACGGCGTAACTCAAAGTTCAAGCAACTTCCAAAATTCATTTACGGACCAAGAT
AGCAAGAAATCAACTTATTCCCTTTCATCGATTTTAACTCTCCCCTCGTCTGAGTATCA
ATCGCATGACGCCTACACTTGTGAGGTTTCTCACAAGTCCTTGACAACCACATTGGTG
AAATCCTTTAGCAAGAATGAATGTGGCGGGGGCGGAAGCGGCGGCGGCGGTTCTGGT
GGAGGAGGCAGCGGAGGAGGCGGCTCCGGGGGAGGGGGAAGCGGCGGTGGAGGGT
CCGGAGGAGGCGGCTCAGGCGGGGGGGGGTCGGGAGGGGGGGGAAGCGGAGGGG
GTGGAAGCGGAGGCGGGGGATCTGGAGGCGGCGGCTCTCAGGTTCAACTCAAGCAG
TCCGGTCCCGGACTGGTTCAGCCATCACAGAACCTCTCTATAACCTGCACTGTCTCCG
GCTTTAGTCTGACCACCTATGGTGTGCACTGGGTGCGGCAGTCTCCTGGCAAGGGGC
TGGATTGGCTGGGTGTTATCTGGGGTGGGGGAAGTACCGATTTTAATGCAGCCTTCAT
GAGCCGCCTATCTATTACCAAGGACAATTCGAAGAGCCAGGTGTTCTTCAAGATGAAC
TCTCTGCAGGCCGACGACACCGCAATTTATTACTGTGCCAAGTCTCTGCTGTCTTCGA
TGGACTACTGGGGCCAAGGGACGTCTGTCACCGTGAGTTCCGCTAGTACGTCCGCGC
CCAAGGTGTACCCCCTCACCAGCTGTTGTGGTGATAAAAGCAGCTCTCGAGTTACAC
TCGGCTGCCTCGTGTCCAGCTATATGCCTGAACCCGTCACCGTCACTTGGAACAGTGG
CGCACTGAAAAGCGGCGTGCACACATTTCCGGCGGTCCTCCAGAGTAGCGGGCTCTA
CTCCCTCTCTAGCATGGTGACTGTGCCTGGGAGCACAAGTGGCCAGACCTTTACCTG
CAATGTGGCCCATCCAGCCTCCAGCACCAAAGTCGATAAGGCTGTGGGAGTTTCCAG
TGACTGCAGCAAGCCTAATAATCAGCATTGCGTGAGGGAACCTAGCGTTTTTATTTTT
CCCCCAAAGCCGAAAGATACCCTGATGATAACAGGAACCCCGGAGGTAACTTGTGTG
GTGGTGAATGTGGGGCATGACAACCCAGAAGTGCAGTTCTCCTGGTTCGTGGATGAT
GTAGAGGTCCATACTGCCCGTACAAAACCCCGGGAGGAGCAGTTTAATTCGACGTAC
AGAGTAGTTTCAGCACTTCCCATCCAGCACCAGGACTGGACAGGCGGAAAGGAATTT
AAGTGCAAAGTGAACATCAAAGGTTTGTCAGCCTCCATCGTACGCATCATTTCTCGTA
GTAAGGGCCCTGCCCGAGAGCCTCAAGTCTATGTACTGGACCCACCTAAAGAAGAGC
TGTCCAAATCTACGGTGAGCGTGACGTGCATGGTCATTGGCTTCTATCCAGAAGATGT
TGACGTGGAGTGGCAGAGGGACAGGCAAACTGAGAGCGAGGACAAATACCGGACC
ACACCTCCGCAGCTTGATGCTGACAGAAGCTATTTCCTGTACTCCAAGCTAAGGGTGG
ACAGAAACTCCTGGCAGCGGGGGGGTACTTACACCTGTGTAGTCATGCATGAGGCCC
TGCACAACCACTACATGCAGAAATCAACATCCAAGAGTGCCGGCAAAGGGGGCGGC
GGCTCTGTTTTCACTTTGGAAGACTTCGTTGGAGATTGGAGACAAACAGCTGGCTAC
AACTTGGATCAGGTGTTGGAGCAGGGCGGTGTGTCTAGCCTCTTCCAGAATCTGGGA
GTGAGCGTTACTCCAATTCAGCGCATCGTCTTAAGCGGGGAGAACGGCCTCAAAATC
GACATTCACGTGATCATCCCCTACGAGGGACTTTCGGGGGATCAGATGGGGCAGATC
GAGAAGATCTTCAAAGTCGTTTATCCTGTGGATGACCACCACTTTAAGGTCATCCTAC
ATTATGGAACTTTAGTCATAGATGGCGTGACACCCAACATGATCGACTATTTTGGGAG
GCCTTATGAAGGCATCGCTGTCTTTGATGGAAAGAAGATTACAGTTACAGGCACCCTG
TGGAATGGGAACAAAATAATAGATGAGCGGCTGATTAACCCTGATGGCTCCTTACTGT
TCCGCGTCACAATCAATGGAGTAACCGGCTGGAGGCTCTGTGAACGCATTCTGGCCG
GCGGGGGTGGGAGCCCAAACAAAGGCAGTGGTACCACATCAGGCACCACGAGACTT
CTCTCTGGACACACCTGTTTCACACTGACCGGCCTGCTTGGAACACTCGTCACTATGG
GTCTTCTGACGTGATAG
GS-linker (contained within ORF)
SEQ ID NO: 19
GGGGGCGGCGGCTCT
Nluc (contained within ORF)
SEQ ID NO: 20
GTTTTCACTTTGGAAGACTTCGTTGGAGATTGGAGACAAACAGCTGGCTACAACTTG
GATCAGGTGTTGGAGCAGGGCGGTGTGTCTAGCCTCTTCCAGAATCTGGGAGTGAGC
GTTACTCCAATTCAGCGCATCGTCTTAAGCGGGGAGAACGGCCTCAAAATCGACATTC
ACGTGATCATCCCCTACGAGGGACTTTCGGGGGATCAGATGGGGCAGATCGAGAAGA
TCTTCAAAGTCGTTTATCCTGTGGATGACCACCACTTTAAGGTCATCCTACATTATGGA
ACTTTAGTCATAGATGGCGTGACACCCAACATGATCGACTATTTTGGGAGGCCTTATG
AAGGCATCGCTGTCTTTGATGGAAAGAAGATTACAGTTACAGGCACCCTGTGGAATG
GGAACAAAATAATAGATGAGCGGCTGATTAACCCTGATGGCTCCTTACTGTTCCGCGT
CACAATCAATGGAGTAACCGGCTGGAGGCTCTGTGAACGCATTCTGGCC
GS-linker (contained within ORF)
SEQ ID NO: 21
GGCGGGGGTGGGAGC
Anchor Sequence (Contained within ORF)
SEQ ID NO: 22
CCAAACAAAGGCAGTGGTACCACATCAGGCACCACGAGACTTCTCTCTGGACACACC
TGTTTCACACTGACCGGCCTGCTTGGAACACTCGTCACTATGGGTCTTCTGACG
3′UTR
SEQ ID NO: 5
GCTCGCTTTCTTGCTGTCCAATTTCTATTAAAGGTTCCTTTGTTCCCTAAGTCCAACTA
CTAAACTGGGGGATATTATGAAGGGCCTTGAGCATCTGGATTCTGCCTAATAAAAAAC
ATTTATTTTCATTGC
Not-I
SEQ ID NO: 6
GCGGCCGC

### IgG Antibodies

### 1. Anti-TF 1.15, Secreted

SEQ ID NO: 26—TF1.15 heavy chain secreted-RNA45-P135 bovIgM-IgG HC

5′UTR
SEQ ID NO: 1
AAGCTTTAATACGACTCACTATAGGGAAATAAGAGAGAAAAGAAGAGTAAGAAGAAA
TATAAGAGCCACC
ORF
SEQ ID NO: 24
ATGGCCGTGCTGGGCCTGCTGTTCTGCCTGGTGACCTTCCCTTCCTGCGTGCTGAGCCAGGT
GCAGCTGAAGCAGGCTGGACCTGGACTGGTGCAGCCTTCCCAGAGCCTGTCCATC
ACCTGCACCGTGTCCGGCTTCAGCCTGTCCAACTACGGAGTGCACTGGGTGCGG
CAGAGCCCTGGCAAGGGCCTGGAGTGGCTGGGACTGATCTGGGGAGGCGGCCGGTC
CGACTACAACGCTGCCTTCATCTCCAGGCTGACCATCAGCAAGGACAACAGCAAGTC
CCAGGTGTTCTTCACCATGAACAGCCTGCAGGTGAACGACACCGGCATCTACTACTG
CGTGCGGAACGGAGGCGACCCTGCTTGGTTCGCTTACTGGGGACAGGGCACCCTGGT
GACCGTGTCCGCCGCTAGCACCTCCGCCCCCAAGGTGTACCCTCTGACCAGCTGCTG
CGGCGACAAGAGCTCCAGCAGGGTGACCCTGGGATGCCTGGTGTCCAGCTACATGCC
CGAGCCTGTGACCGTGACCTGGAACTCCGGCGCCCTGAAGAGCGGAGTGCACACCT
TCCCTGCCGTGCTGCAGTCCAGCGGACTGTACTCCCTGTCCAGCATGGTGACCGTGC
CTGGAAGCACCTCCGGACAGACCTTCACCTGCAACGTGGCTCACCCTGCTTCCAGCA
CCAAGGTGGACAAGGCCGTGGGCGTGTCCAGCGACTGCTCCAAGCCCAACAACCAG
CACTGCGTGCGGGAGCCTAGCGTGTTCATCTTCCCCCCTAAGCCCAAGGACACCCTG
ATGATCACCGGAACCCCTGAGGTGACCTGCGTGGTGGTGAACGTGGGCCACGACAA
CCCCGAGGTGCAGTTCAGCTGGTTCGTGGACGACGTGGAGGTGCACACCGCTAGGA
CCAAGCCTAGGGAGGAGCAGTTCAACTCCACCTACAGGGTGGTGAGCGCCCTGCCTA
TCCAGCACCAGGACTGGACCGGAGGCAAGGAGTTCAAGTGCAAGGTGAACATCAAG
GGCCTGAGCGCCTCCATCGTGCGCATCATCAGCAGATCCAAGGGCCCCGCCAGAGAG
CCTCAGGTGTACGTGCTGGACCCCCCTAAGGAGGAGCTGAGCAAGTCCACCGTGTCC
GTGACCTGCATGGTCATCGGCTTCTACCCTGAGGACGTGGACGTGGAGTGGCAGAGG
GACAGGCAGACCGAGAGCGAGGACAAGTACAGGACCACCCCTCCTCAGCTGGACGC
TGACAGATCCTACTTCCTGTACAGCAAGCTGCGCGTGGACAGAAACAGCTGGCAGAG
GGGAGGAACCTACACCTGCGTGGTCATGCACGAGGCCCTGCACAACCACTACATGCA
GAAGAGCACCTCCAAGAGCGCCGGCAAGTGATGA
3′UTR
SEQ ID NO: 25
GCTGCCTTCTGCGGGGCTTGCCTTCTGGCCATGCCCTTCTTCTCTCCCTTGCACCTGTACCT
CTTGGTCTTTGAATAAAGCCTGAGTAGGAAG
Not-I
SEQ ID NO: 6
GCGGCCGC
SEQ ID NO: 28-TF1.15 light chain-RNA46-P135 bovIgM-IgG LC
5′UTR
SEQ ID NO: 1
AAGCTTTAATACGACTCACTATAGGGAAATAAGAGAGAAAAGAAGAGTAAGAAGAAA
TATAAGAGCCACC
ORF
SEQ ID NO: 27
ATGGCCTGGATCAGCCTGATCCTGTCCCTGCTGGCCCTGAGCTCCGGAGCCATCAGCC
AGGCTGTGGTGACCCAGGAGAGCGCCCTGACCACCTCCCCTGGAGAGACCGTGACC
CTGACCTGCCGCAGCTCCACCGGAGCTGTGACCACCTCCAACTACGCCAACTGGGTG
CAGGAGAAGCCTGACCACCTGTTCACCGGCCTGATCGGAGGAACCAACAACAGGGC
TCCTGGAGTGCCTGCCAGGTTCTCCGGCAGCCTGATCGGCGACAAGGCCGCCCTGAC
CATCACCGGAGCTCAGACCGAGGACGAGGCCATCTACTTCTGCGCCCTGTGGTACAG
CAACCTGTGGGTGTTCGGAGGAGGAACCAAGCTGACCGTGCTGCGGAGCGACGCTG
AGCCTTCCGTGTTCCTGTTCAAGCCTAGCGACGAGCAGCTGAAGACCGGCACCGTGT
CCGTGGTGTGCCTGGTGAACGACTTCTACCCCAAGGACATCAACGTGAAGTGGAAGG
TGGACGGCGTGACCCAGAGCTCCAGCAACTTCCAGAACAGCTTCACCGACCAGGAC
TCCAAGAAGAGCACCTACTCCCTGTCCAGCATCCTGACCCTGCCTTCCAGCGAGTAC
CAGTCCCACGACGCCTACACCTGCGAGGTGAGCCACAAGTCCCTGACCACCACCCTG
GTGAAGTCCTTCAGCAAGAACGAGTGCTGATGA
3′UTR
SEQ ID NO: 25
GCTGCCTTCTGCGGGGCTTGCCTTCTGGCCATGCCCTTCTTCTCTCCCTTGCACCTGTA
CCTCTTGGTCTTTGAATAAAGCCTGAGTAGGAAG
Not-I
SEQ ID NO: 6
GCGGCCGC

### 2. Anti-TF 1.15, Anchored

SEQ ID NO: 31—TF1.15 heavy chain anchored-RNA188-P135 bovIgM-IgG HC GPI

5′UTR
SEQ ID NO: 1
AAGCTTTAATACGACTCACTATAGGGAAATAAGAGAGAAAAGAAGAGTAAGAAGAAA
TATAAGAGCCACC
ORF
SEQ ID NO: 29
ATGGCCGTGCTGGGCCTGCTGTTCTGCCTGGTGACCTTCCCTTCCTGCGTGCTGAGCCAGGT
GCAGCTGAAGCAGGCTGGACCTGGACTGGTGCAGCCTTCCCAGAGCCTGTCC
ATCACCTGCACCGTGTCCGGCTTCAGCCTGTCCAACTACGGAGTGCACTGGGTGCGG
CAGAGCCCTGGCAAGGGCCTGGAGTGGCTGGGACTGATCTGGGGAGGCGGCCGGTC
CGACTACAACGCTGCCTTCATCTCCAGGCTGACCATCAGCAAGGACAACAGCAAGTC
CCAGGTGTTCTTCACCATGAACAGCCTGCAGGTGAACGACACCGGCATCTACTACTG
CGTGCGGAACGGAGGCGACCCTGCTTGGTTCGCTTACTGGGGACAGGGCACCCTGGT
GACCGTGTCCGCCGCTAGCACCTCCGCCCCCAAGGTGTACCCTCTGACCAGCTGCTG
CGGCGACAAGAGCTCCAGCAGGGTGACCCTGGGATGCCTGGTGTCCAGCTACATGCC
CGAGCCTGTGACCGTGACCTGGAACTCCGGCGCCCTGAAGAGCGGAGTGCACACCT
TCCCTGCCGTGCTGCAGTCCAGCGGACTGTACTCCCTGTCCAGCATGGTGACCGTGC
CTGGAAGCACCTCCGGACAGACCTTCACCTGCAACGTGGCTCACCCTGCTTCCAGCA
CCAAGGTGGACAAGGCCGTGGGCGTGTCCAGCGACTGCTCCAAGCCCAACAACCAG
CACTGCGTGCGGGAGCCTAGCGTGTTCATCTTCCCCCCTAAGCCCAAGGACACCCTG
ATGATCACCGGAACCCCTGAGGTGACCTGCGTGGTGGTGAACGTGGGCCACGACAA
CCCCGAGGTGCAGTTCAGCTGGTTCGTGGACGACGTGGAGGTGCACACCGCTAGGA
CCAAGCCTAGGGAGGAGCAGTTCAACTCCACCTACAGGGTGGTGAGCGCCCTGCCTA
TCCAGCACCAGGACTGGACCGGAGGCAAGGAGTTCAAGTGCAAGGTGAACATCAAG
GGCCTGAGCGCCTCCATCGTGCGCATCATCAGCAGATCCAAGGGCCCCGCCAGAGAG
CCTCAGGTGTACGTGCTGGACCCCCCTAAGGAGGAGCTGAGCAAGTCCACCGTGTCC
GTGACCTGCATGGTCATCGGCTTCTACCCTGAGGACGTGGACGTGGAGTGGCAGAGG
GACAGGCAGACCGAGAGCGAGGACAAGTACAGGACCACCCCTCCTCAGCTGGACGC
TGACAGATCCTACTTCCTGTACAGCAAGCTGCGCGTGGACAGAAACAGCTGGCAGAG
GGGAGGAACCTACACCTGCGTGGTCATGCACGAGGCCCTGCACAACCACTACATGCA
GAAGAGCACCTCCAAGAGCGCCGGCAAGCACGAGACAACACCTAACAAGGGCAGC
GGAACTACCAGCGGCACCACCCGGCTGCTGAGCGGCCACACCTGCTTCACCCTGACA
GGCCTGCTGGGCACCCTGGTCACAATGGGCCTGCTCACCTGATGA
Anchor Sequence (contained within ORF)
SEQ ID NO: 30
CACGAGACAACACCTAACAAGGGCAGCGGAACTACCAGCGGCACCACCCGGCTGCT
GAGCGGCCACACCTGCTTCACCCTGACAGGCCTGCTGGGCACCCTGGTCACAATGGG
CCTGCTCACC
3′UTR
SEQ ID NO: 25
GCTGCCTTCTGCGGGGCTTGCCTTCTGGCCATGCCCTTCTTCTCTCCCTTGCACCTGTA
CCTCTTGGTCTTTGAATAAAGCCTGAGTAGGAAG
Not-I
SEQ ID NO: 6
GCGGCCGC
SEQ ID NO: 35-TF1.15 light chain NanoLuc-RNA189-P135 bovIgM-IgG LC NLuc
5′UTR
SEQ ID NO: 1
AAGCTTTAATACGACTCACTATAGGGAAATAAGAGAGAAAAGAAGAGTAAGAAGAAA
TATAAGAGCCACC
ORF
SEQ ID NO: 32
ATGGCCTGGATCAGCCTGATCCTGTCCCTGCTGGCCCTGAGCTCCGGAGCCATCAGCC
AGGCTGTGGTGACCCAGGAGAGCGCCCTGACCACCTCCCCTGGAGAGACCGTGACC
CTGACCTGCCGCAGCTCCACCGGAGCTGTGACCACCTCCAACTACGCCAACTGGGTG
CAGGAGAAGCCTGACCACCTGTTCACCGGCCTGATCGGAGGAACCAACAACAGGGC
TCCTGGAGTGCCTGCCAGGTTCTCCGGCAGCCTGATCGGCGACAAGGCCGCCCTGAC
CATCACCGGAGCTCAGACCGAGGACGAGGCCATCTACTTCTGCGCCCTGTGGTACAG
CAACCTGTGGGTGTTCGGAGGAGGAACCAAGCTGACCGTGCTGCGGAGCGACGCTG
AGCCTTCCGTGTTCCTGTTCAAGCCTAGCGACGAGCAGCTGAAGACCGGCACCGTGT
CCGTGGTGTGCCTGGTGAACGACTTCTACCCCAAGGACATCAACGTGAAGTGGAAGG
TGGACGGCGTGACCCAGAGCTCCAGCAACTTCCAGAACAGCTTCACCGACCAGGAC
TCCAAGAAGAGCACCTACTCCCTGTCCAGCATCCTGACCCTGCCTTCCAGCGAGTAC
CAGTCCCACGACGCCTACACCTGCGAGGTGAGCCACAAGTCCCTGACCACCACCCTG
GTGAAGTCCTTCAGCAAGAACGAGTGCGGAGGAGGAGGAAGCGTGTTCACCCTGGA
GGACTTTGTGGGCGATTGGAGGCAGACCGCCGGCTATAACCTGGACCAGGTGCTGGA
GCAGGGAGGCGTGTCCTCTCTGTTTCAGAATCTGGGCGTGTCCGTGACCCCAATCCA
GAGGATCGTGCTGTCTGGCGAGAACGGCCTGAAGATCGACATCCACGTGATCATCCC
TTACGAGGGCCTGTCCGGCGATCAGATGGGCCAGATCGAGAAGATCTTCAAGGTGGT
GTACCCAGTGGACGATCACCACTTCAAAGTGATCCTGCACTATGGCACCCTGGTCATC
GACGGCGTGACCCCAAATATGATCGATTACTTCGGCAGACCCTATGAGGGCATCGCCG
TGTTTGATGGCAAGAAGATCACAGTGACCGGCACACTGTGGAACGGCAATAAGATCA
TCGACGAGCGGCTGATCAACCCCGATGGCAGCCTGCTGTTCAGAGTGACAATCAATG
GAGTGACCGGATGGAGGCTGTGCGAGAGAATCCTGGCCTGATGA
GS-LINKER (contained within ORF)
SEQ ID NO: 33
GGAGGAGGAGGAAGC
Nluc (contained within ORF)
SEQ ID NO: 34
GTGTTCACCCTGGAGGACTTTGTGGGCGATTGGAGGCAGACCGCCGGCTATAACCTG
GACCAGGTGCTGGAGCAGGGAGGCGTGTCCTCTCTGTTTCAGAATCTGGGCGTGTCC
GTGACCCCAATCCAGAGGATCGTGCTGTCTGGCGAGAACGGCCTGAAGATCGACATC
CACGTGATCATCCCTTACGAGGGCCTGTCCGGCGATCAGATGGGCCAGATCGAGAAG
ATCTTCAAGGTGGTGTACCCAGTGGACGATCACCACTTCAAAGTGATCCTGCACTATG
GCACCCTGGTCATCGACGGCGTGACCCCAAATATGATCGATTACTTCGGCAGACCCTA
TGAGGGCATCGCCGTGTTTGATGGCAAGAAGATCACAGTGACCGGCACACTGTGGAA
CGGCAATAAGATCATCGACGAGCGGCTGATCAACCCCGATGGCAGCCTGCTGTTCAG
AGTGACAATCAATGGAGTGACCGGATGGAGGCTGTGCGAGAGAATCCTGGCC
3′UTR
SEQ ID NO: 25
GCTGCCTTCTGCGGGGCTTGCCTTCTGGCCATGCCCTTCTTCTCTCCCTTGCACCTGTA
CCTCTTGGTCTTTGAATAAAGCCTGAGTAGGAAG
Not-I
SEQ ID NO: 6
GCGGCCGC

### 3. Anti-TF 1.17, Secreted

SEQ ID NO: 37-TF1.17 heavy chain secreted-RNA31-
P137 bovIgM-IgG HC
5′UTR
SEQ ID NO: 1
AAGCTTTAATACGACTCACTATAGGGAAATAAGAGAGAAAAGAAGAGTA
AGAAGAAATATAAGAGCCACC
ORF
SEQ ID NO: 36
AAGCTTTAATACGACTCACTATAGGGAAATAAGAGAGAAAAGAAGAGTA
AGAAGAAATATAAGAGCCACC
3′UTR
SEQ ID NO: 25
GCTGCCTTCTGCGGGGCTTGCCTTCTGGCCATGCCCTTCTTCTCTCCCT
TGCACCTGTACCTCTTGGTCTTTGAATAAAGCCTGAGTAGGAAG
Not-I
SEQ ID NO: 6
GCGGCCGC
SEQ ID NO: 39-TF1.17 light chain-RNA32-P137
bovIgM-IgG LC
5′UTR
SEQ ID NO: 1
AAGCTTTAATACGACTCACTATAGGGAAATAAGAGAGAAAAGAAGAGTA
AGAAGAAATATAAGAGCCACC
ORF
SEQ ID NO: 38
ATGGACAGCCAGGCCCAGGTGCTGATGCTGCTGCTGCTGTGGGTGTCCG
GAACCTGCGGCGACATCGTGATGTCCCAGAGCCCTAGCTCCCTGGCCGT
GAGCGTGGGAGAGAAGGTGACCATGAGCTGCAAGAGCTCCCAGTCCCTG
CTGTACAGCTCCAACCAGAAGAACTACCTGGCCTGGTACCAGCAGAAGC
CCGGCCAGAGCCCTAAGCTGCTGATCTACTGGGCTAGCACCAGGGAGTC
CGGAGTGCCTGACAGGTTCACCGGATCCGGAAGCGGAACCGACTTCACC
CTGACTATCAGCTCCGTGAAGGCCGAGGACCTGGCCGTGTACTACTGCC
AGCAGTACTACAACTACCCTTTCACCTTCGGCGGCGGCACCAACCTGGA
GATCAAGCGGAGCGACGCTGAGCCTTCCGTGTTCCTGTTCAAGCCTAGC
GACGAGCAGCTGAAGACCGGCACCGTGTCCGTGGTGTGCCTGGTGAACG
ACTTCTACCCCAAGGACATCAACGTGAAGTGGAAGGTGGACGGCGTGAC
CCAGAGCTCCAGCAACTTCCAGAACAGCTTCACCGACCAGGACTCCAAG
AAGAGCACCTACTCCCTGTCCAGCATCCTGACCCTGCCTTCCAGCGAGT
ACCAGTCCCACGACGCCTACACCTGCGAGGTGAGCCACAAGTCCCTGAC
CACCACCCTGGTGAAGTCCTTCAGCAAGAACGAGTGCTGATGA
3′UTR
SEQ ID NO: 25
GCTGCCTTCTGCGGGGCTTGCCTTCTGGCCATGCCCTTCTTCTCTCCCT
TGCACCTGTACCTCTTGGTCTTTGAATAAAGCCTGAGTAGGAAG
Not-I
SEQ ID NO: 6
GCGGCCGC

### 4. Anti-TF 1.17, Anchored

SEQ ID NO: 41—TF1.17 heavy chain anchored-RNA190-P137 bovIgM-IgG HC GPI

5′UTR
SEQ ID NO: 1
AAGCTTTAATACGACTCACTATAGGGAAATAAGAGAGAAAAGAAGAGTAAGAAGAAA
TATAAGAGCCACC
ORF
SEQ ID NO: 40
ATGGCCGTGCTGGTGCTGCTGCTGTGCCTGGTGACCTTCCCTAGCTGCGTGCTGTCCC
AGGTGCAGCTGAAGCAGAGCGGACCTGGACTGGTGCAGCCTAGCCAGAACCTGTCC
ATCACCTGCACCGTGAGCGGCTTCTCCCTGACCACCTACGGAGTGCACTGGGTGCGC
CAGAGCCCTGGCAAGGGCCTGGACTGGCTGGGCGTGATCTGGGGAGGAGGATCCAC
CGACTTCAACGCTGCCTTCATGAGCAGACTGTCCATCACCAAGGACAACTCCAAGAG
CCAGGTGTTCTTCAAGATGAACAGCCTGCAGGCCGACGACACCGCCATCTACTACTG
CGCCAAGAGCCTGCTGAGCTCCATGGACTACTGGGGACAGGGCACCTCCGTGACCGT
GAGCTCCGCCTCCACCAGCGCCCCTAAGGTGTACCCTCTGACCTCCTGCTGCGGCGA
CAAGAGCTCCAGCAGGGTGACCCTGGGATGCCTGGTGTCCAGCTACATGCCCGAGCC
TGTGACCGTGACCTGGAACAGCGGCGCCCTGAAGTCCGGAGTGCACACCTTCCCTGC
CGTGCTGCAGTCCAGCGGACTGTACAGCCTGTCCAGCATGGTGACCGTGCCTGGATC
CACCAGCGGACAGACCTTCACCTGCAACGTGGCTCACCCTGCTTCCAGCACCAAGGT
GGACAAGGCCGTGGGCGTGTCCAGCGACTGCAGCAAGCCCAACAACCAGCACTGCG
TGAGAGAGCCTTCCGTGTTCATCTTCCCCCCTAAGCCCAAGGACACCCTGATGATCAC
CGGAACCCCTGAGGTGACCTGCGTGGTGGTGAACGTGGGCCACGACAACCCCGAGG
TGCAGTTCTCCTGGTTCGTGGACGACGTGGAGGTGCACACCGCTAGGACCAAGCCTA
GGGAGGAGCAGTTCAACAGCACCTACAGGGTGGTGTCCGCCCTGCCTATCCAGCACC
AGGACTGGACCGGAGGCAAGGAGTTCAAGTGCAAGGTGAACATCAAGGGCCTGTCC
GCCAGCATCGTGCGCATCATCTCCAGAAGCAAGGGACCTGCTAGGGAGCCTCAGGTG
TACGTGCTGGACCCCCCTAAGGAGGAGCTGTCCAAGAGCACCGTGAGCGTGACCTG
CATGGTCATCGGCTTCTACCCTGAGGACGTGGACGTGGAGTGGCAGAGGGACAGGC
AGACCGAGTCCGAGGACAAGTACAGGACCACCCCTCCTCAGCTGGACGCTGACAGG
AGCTACTTCCTGTACTCCAAGCTGCGCGTGGACAGAAACAGCTGGCAGAGGGGAGG
AACCTACACCTGCGTGGTCATGCACGAGGCCCTGCACAACCACTACATGCAGAAGTC
CACCAGCAAGTCCGCCGGCAAGCACGAGACAACACCTAACAAGGGCAGCGGAACTA
CCAGCGGCACCACCCGGCTGCTGAGCGGCCACACCTGCTTCACCCTGACAGGCCTGC
TGGGCACCCTGGTCACAATGGGCCTGCTCACCTGATGA
Anchor Sequence (contained within ORF)
SEQ ID NO: 30
CACGAGACAACACCTAACAAGGGCAGCGGAACTACCAGCGGCACCACCCGGCTGCT
GAGCGGCCACACCTGCTTCACCCTGACAGGCCTGCTGGGCACCCTGGTCACAATGGG
CCTGCTCACC
3′UTR
SEQ ID NO: 25
GCTGCCTTCTGCGGGGCTTGCCTTCTGGCCATGCCCTTCTTCTCTCCCTTGCACCTGTA
CCTCTTGGTCTTTGAATAAAGCCTGAGTAGGAAG
Not-I
SEQ ID NO: 6
GCGGCCGC
SEQ ID NO: 44-TF1.17 light chain NanoLuc-RNA191-P137 bovIgM-IgG LC NLuc
5′UTR
SEQ ID NO: 1
AAGCTTTAATACGACTCACTATAGGGAAATAAGAGAGAAAAGAAGAGTAAGAAGAAA
TATAAGAGCCACC
ORF
SEQ ID NO: 42
ATGGACAGCCAGGCCCAGGTGCTGATGCTGCTGCTGCTGTGGGTGTCCGGAACCTGC
GGCGACATCGTGATGTCCCAGAGCCCTAGCTCCCTGGCCGTGAGCGTGGGAGAGAAG
GTGACCATGAGCTGCAAGAGCTCCCAGTCCCTGCTGTACAGCTCCAACCAGAAGAAC
TACCTGGCCTGGTACCAGCAGAAGCCCGGCCAGAGCCCTAAGCTGCTGATCTACTGG
GCTAGCACCAGGGAGTCCGGAGTGCCTGACAGGTTCACCGGATCCGGAAGCGGAAC
CGACTTCACCCTGACTATCAGCTCCGTGAAGGCCGAGGACCTGGCCGTGTACTACTG
CCAGCAGTACTACAACTACCCTTTCACCTTCGGCGGCGGCACCAACCTGGAGATCAA
GCGGAGCGACGCTGAGCCTTCCGTGTTCCTGTTCAAGCCTAGCGACGAGCAGCTGAA
GACCGGCACCGTGTCCGTGGTGTGCCTGGTGAACGACTTCTACCCCAAGGACATCAA
CGTGAAGTGGAAGGTGGACGGCGTGACCCAGAGCTCCAGCAACTTCCAGAACAGCT
TCACCGACCAGGACTCCAAGAAGAGCACCTACTCCCTGTCCAGCATCCTGACCCTGC
CTTCCAGCGAGTACCAGTCCCACGACGCCTACACCTGCGAGGTGAGCCACAAGTCCC
TGACCACCACCCTGGTGAAGTCCTTCAGCAAGAACGAGTGCGGAGGAGGAGGAAGC
GTGTTCACCCTGGAGGACTTTGTGGGCGATTGGAGGCAGACCGCCGGCTATAACCTG
GACCAGGTGCTGGAGCAGGGAGGCGTGTCCTCTCTGTTTCAGAATCTGGGCGTGTCC
GTGACCCCAATCCAGAGGATCGTGCTGTCTGGCGAGAACGGCCTGAAGATCGACATC
CACGTGATCATCCCTTACGAGGGCCTGTCCGGCGATCAGATGGGCCAGATCGAGAAG
ATCTTCAAGGTGGTGTACCCAGTGGACGATCACCACTTCAAAGTGATCCTGCACTATG
GCACCCTGGTCATCGACGGCGTGACCCCAAATATGATCGATTACTTCGGCAGACCCTA
TGAGGGCATCGCCGTGTTTGATGGCAAGAAGATCACAGTGACCGGCACACTGTGGAA
CGGCAATAAGATCATCGACGAGCGGCTGATCAACCCCGATGGCAGCCTGCTGTTCAG
AGTGACAATCAATGGAGTGACCGGATGGAGGCTGTGCGAGAGAATCCTGGCCTGATGA
GS-LINKER (contained within ORF)
SEQ ID NO: 33
GGAGGAGGAGGAAGC
Nluc (contained within ORF)
SEQ ID NO: 43
GTGTTCACCCTGGAGGACTTTGTGGGCGATTGGAGGCAGACCGCCGGCTATAACCTG
GACCAGGTGCTGGAGCAGGGAGGCGTGTCCTCTCTGTTTCAGAATCTGGGCGTGTCC
GTGACCCCAATCCAGAGGATCGTGCTGTCTGGCGAGAACGGCCTGAAGATCGACATC
CACGTGATCATCCCTTACGAGGGCCTGTCCGGCGATCAGATGGGCCAGATCGAGAAG
ATCTTCAAGGTGGTGTACCCAGTGGACGATCACCACTTCAAAGTGATCCTGCACTATG
GCACCCTGGTCATCGACGGCGTGACCCCAAATATGATCGATTACTTCGGCAGACCCTA
TGAGGGCATCGCCGTGTTTGATGGCAAGAAGATCACAGTGACCGGCACACTGTGGAA
CGGCAATAAGATCATCGACGAGCGGCTGATCAACCCCGATGGCAGCCTGCTGTTCAG
AGTGACAATCAATGGAGTGACCGGATGGAGGCTGTGCGAGAGAATCCTGGCC
3′UTR
SEQ ID NO: 25
GCTGCCTTCTGCGGGGCTTGCCTTCTGGCCATGCCCTTCTTCTCTCCCTTGCACCTGTA
CCTCTTGGTCTTTGAATAAAGCCTGAGTAGGAAG
Not-I
SEQ ID NO: 6
GCGGCCGC

What is claimed is:

1. A synthetic mRNA construct encoding for an antibody against a cell surface antigen of Tritichomonas foetus (Tf), said synthetic mRNA construct comprising a peptide sequence selected from the group consisting of SEQ ID NO: 7, SEQ ID NO: 13, SEQ ID NO: 17, SEQ ID NO: 23, SEQ ID NO: 26, SEQ ID NO: 28, SEQ ID NO: 31, SEQ ID NO: 35, SEQ ID NO: 37, SEQ ID NO: 39, SEQ ID NO: 41, and SEQ ID NO: 44 for administration to a mammal to induce expression of the antibody.

2. The synthetic mRNA construct of claim 1, further comprising a secretion signal peptide.

3. The synthetic mRNA construct of claim 1, further comprising a membrane anchor signal peptide.

4. The synthetic mRNA construct of claim 2, wherein the membrane anchor signal peptide has a peptide sequence selected from the group consisting of SEQ ID NO: 12, SEQ ID NO: 22, and SEQ ID NO: 30.

5. The synthetic mRNA construct of claim 1, wherein the mammal is selected from the group consisting of a bovine and a cat.

6. The synthetic mRNA construct of claim 1, wherein the mammal is a male bovine.

7. A method for treating Tritichomonas foetus in a male bovine, comprising a step of administering the synthetic mRNA construct of claim 1 to a preputial tissue of a male bovine.

8. The method of claim 7, wherein the mRNA is administered to a preputial fornix of the male bovine.

9. The method of claim 7, comprising administration of a dose of 0.5 mg to 10 mg of the mRNA construct to the preputial tissue.

10. The method of claim 7, comprising administration of a dose of 1.0 mg to about 5 mg of the mRNA construct to the preputial tissue.

11. The method of claim 7, wherein the mRNA construct is administered as an aerosol.

12. The method of claim 11, wherein the mRNA is administered using an atomization device.

13. The method of claim 7, wherein the mRNA construct is suspended in a carrier.

14. The method of claim 13, wherein the carrier is selected from the group consisting of molecular grade water, lipid nanoparticles, liposomes, poly-based nanoparticles, cell-penetrating peptides, extracellular vesicles, and combinations thereof.


SEQUENCE LISTING (44 sequences)

SEQ ID NO: 1 - DNA, 70 bp, synthetic construct
  source 1..70: mol_type = other DNA
aagctttaatacgactcactatagggaaataagagagaaaagaagagtaagaagaaatataagagccacc

SEQ ID NO: 2 - DNA, 2748 bp, synthetic construct
  source 1..2748: mol_type = other DNA
atgaaatgggtcacttttatcagcctcttgtttctgttcagcagtgcctattctcaagccgtggttacgcaggaaagtgccctgacaacctcacctggggaaacagtaactctgacctgcaggagctcaactggagcagttacaacttccaactacgccaactgggtgcaggagaagcccgaccatctgtttacaggcctgattggcggcaccaacaaccgggcccctggtgtacccgctcgcttttcaggtagccttataggagataaagctgcgctaacgatcactggtgcacagacagaggatgaagccatctatttctgcgcgttgtggtactcaaatctgtgggttttcggcggcggcaccaagctaactgtgctgcgctctgatgctgagcctagcgttttcctgtttaaaccaagcgacgaacagctcaagactgggacagtatctgttgtgtgtcttgtgaacgacttttatcctaaggacatcaatgtgaagtggaaggtggatggcgtgactcagtcatcttctaacttccagaatagcttcacagatcaggatagcaagaaaagcacatatagccttagttctatccttacgctaccctccagtgagtaccagtcccatgatgcctacacatgtgaagttagccacaagtctcttacaacaaccctggtaaagagcttcagtaaaaatgaatgtggcggtggaggatctggagggggcgggtccgggggtgggggatccggaggaggcgggtcaggtggtggtggcagcggaggcggcggatctggtggcggcggctccggcgggggtggctccgggggaggaggctcgggcggtggtgggagtggaggaggtggctccggcggcggcggttcccaagtccagttaaagcaggcaggcccagggctcgttcaaccgagtcaatctctgtctataacgtgcaccgtcagcggatttagtctctccaattatggagtccactgggtgcgccagtccccgggaaaggggctggaatggctgggcttaatctggggcggggggcgaagtgattataatgcagcctttatttcccgactcaccattagcaaggataactctaagagccaggtcttcttcaccatgaattccctgcaggtcaatgacacgggaatctactactgtgtccgcaatggtggcgaccccgcttggttcgcttactggggccagggcacgctcgtaaccgtgtccgcagctagcacaagcgcccccaaagtatatcctttgacctcttgctgcggggacaagtcaagctcaagagtgacattgggctgcctggtctcaagttatatgcctgagccggtgactgttacctggaatagtggtgcgctgaaatctggggtgcacaccttccctgcggtgctccagagcagcggcctctactctctctcctccatggttaccgtcccaggaagtacctccgggcagacgtttacctgcaacgtggcccaccctgcaagcagtacaaaagtcgacaaggctgtgggcgtttcttcagactgtagtaagcctaacaatcagcattgtgtgagggagccatcggtgttcatctttcccccgaagccaaaagacactctaatgattactggtacacctgaagtcacctgtgtcgtagtcaacgttggccatgacaatccagaggtccagttttcgtggtttgtggacgacgtcgaggtgcatacagccagaaccaaaccccgggaggagcagttcaactcaacctaccgggtcgtctcggccctccctatccagcaccaggactggacggggggcaaggagtttaagtgcaaggttaacatcaaaggcttgtcagcgagtatagtgcggatcatatccagatcaaaaggaccagctcgagagccccaggtgtatgttttggacccaccaaaggaagagctgagcaaaagcacagtgtctgtcacatgcatggtgatcggcttctaccccgaagatgtggacgtggagtggcagagagaccggcagaccgagagcgaagacaagtacaggaccaccccgccacagctggatgcagatcgttcctacttcctgtacagcaagctgcgcgtggacaggaactcctggcagcgggggggaacttacacttgcgtggtcatgcacgaagccctccacaaccactacatgcagaagtctacctctaaaagcgccggaaaagggggcggaggctcggtattcaccttggaagatttcgtgggtgattggaggcaaaccgctggctacaacctggaccaagtgctggagcaagggggcgtttcaagtctttttcaaaacctcggtgtgtccgtgacacccattcagaggatcgtactgtcgggggagaatgggctgaagatagatattcatgtgataatcccctatgaaggactcagtggggaccagatgggccaaattgaaaaaatcttcaaagtggtatatccagttgatgaccaccacttcaaggtgattctgcactatgggaccctggttattgacggtgtcacgcccaacatgatcgactactttgggcggccctacgaggggattgccgtgtttgatggaaagaaaataacagtgaccgggactttatggaatggcaacaaaattattgatgagcgactgatcaaccctgatggatctctacttttccgtgtaactattaatggagtgactggctggagactgtgtgagagaatccttgcatgatag

SEQ ID NO: 3 - DNA, 15 bp, synthetic construct
  source 1..15: mol_type = other DNA
gggggcggaggctcg

SEQ ID NO: 4 - DNA, 510 bp, synthetic construct
  source 1..510: mol_type = other DNA
gtattcaccttggaagatttcgtgggtgattggaggcaaaccgctggctacaacctggaccaagtgctggagcaagggggcgtttcaagtctttttcaaaacctcggtgtgtccgtgacacccattcagaggatcgtactgtcgggggagaatgggctgaagatagatattcatgtgataatcccctatgaaggactcagtggggaccagatgggccaaattgaaaaaatcttcaaagtggtatatccagttgatgaccaccacttcaaggtgattctgcactatgggaccctggttattgacggtgtcacgcccaacatgatcgactactttgggcggccctacgaggggattgccgtgtttgatggaaagaaaataacagtgaccgggactttatggaatggcaacaaaattattgatgagcgactgatcaaccctgatggatctctacttttccgtgtaactattaatggagtgactggctggagactgtgtgagagaatccttgca

SEQ ID NO: 5 - DNA, 132 bp, synthetic construct
  source 1..132: mol_type = other DNA
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 6 - skipped

SEQ ID NO: 7 - DNA, 3483 bp, synthetic construct
  source 1..3483: mol_type = other DNA
aagctttaatacgactcactatagggaaataagagagaaaagaagagtaagaagaaatataagagccaccatgaaatgggtcacttttatcagcctcttgtttctgttcagcagtgcctattctcaagccgtggttacgcaggaaagtgccctgacaacctcacctggggaaacagtaactctgacctgcaggagctcaactggagcagttacaacttccaactacgccaactgggtgcaggagaagcccgaccatctgtttacaggcctgattggcggcaccaacaaccgggcccctggtgtacccgctcgcttttcaggtagccttataggagataaagctgcgctaacgatcactggtgcacagacagaggatgaagccatctatttctgcgcgttgtggtactcaaatctgtgggttttcggcggcggcaccaagctaactgtgctgcgctctgatgctgagcctagcgttttcctgtttaaaccaagcgacgaacagctcaagactgggacagtatctgttgtgtgtcttgtgaacgacttttatcctaaggacatcaatgtgaagtggaaggtggatggcgtgactcagtcatcttctaacttccagaatagcttcacagatcaggatagcaagaaaagcacatatagccttagttctatccttacgctaccctccagtgagtaccagtcccatgatgcctacacatgtgaagttagccacaagtctcttacaacaaccctggtaaagagcttcagtaaaaatgaatgtggcggtggaggatctggagggggcgggtccgggggtgggggatccggaggaggcgggtcaggtggtggtggcagcggaggcggcggatctggtggcggcggctccggcgggggtggctccgggggaggaggctcgggcggtggtgggagtggaggaggtggctccggcggcggcggttcccaagtccagttaaagcaggcaggcccagggctcgttcaaccgagtcaatctctgtctataacgtgcaccgtcagcggatttagtctctccaattatggagtccactgggtgcgccagtccccgggaaaggggctggaatggctgggcttaatctggggcggggggcgaagtgattataatgcagcctttatttcccgactcaccattagcaaggataactctaagagccaggtcttcttcaccatgaattccctgcaggtcaatgacacgggaatctactactgtgtccgcaatggtggcgaccccgcttggttcgcttactggggccagggcacgctcgtaaccgtgtccgcagctagcacaagcgcccccaaagtatatcctttgacctcttgctgcggggacaagtcaagctcaagagtgacattgggctgcctggtctcaagttatatgcctgagccggtgactgttacctggaatagtggtgcgctgaaatctggggtgcacaccttccctgcggtgctccagagcagcggcctctactctctctcctccatggttaccgtcccaggaagtacctccgggcagacgtttacctgcaacgtggcccaccctgcaagcagtacaaaagtcgacaaggctgtgggcgtttcttcagactgtagtaagcctaacaatcagcattgtgtgagggagccatcggtgttcatctttcccccgaagccaaaagacactctaatgattactggtacacctgaagtcacctgtgtcgtagtcaacgttggccatgacaatccagaggtccagttttcgtggtttgtggacgacgtcgaggtgcatacagccagaaccaaaccccgggaggagcagttcaactcaacctaccgggtcgtctcggccctccctatccagcaccaggactggacggggggcaaggagtttaagtgcaaggttaacatcaaaggcttgtcagcgagtatagtgcggatcatatccagatcaaaaggaccagctcgagagccccaggtgtatgttttggacccaccaaaggaagagctgagcaaaagcacagtgtctgtcacatgcatggtgatcggcttctaccccgaagatgtggacgtggagtggcagagagaccggcagaccgagagcgaagacaagtacaggaccaccccgccacagctggatgcagatcgttcctacttcctgtacagcaagctgcgcgtggacaggaactcctggcagcgggggggaacttacacttgcgtggtcatgcacgaagccctccacaaccactacatgcagaagtctacctctaaaagcgccggaaaagggggcggaggctcggtattcaccttggaagatttcgtgggtgattggaggcaaaccgctggctacaacctggaccaagtgctggagcaagggggcgtttcaagtctttttcaaaacctcggtgtgtccgtgacacccattcagaggatcgtactgtcgggggagaatgggctgaagatagatattcatgtgataatcccctatgaaggactcagtggggaccagatgggccaaattgaaaaaatcttcaaagtggtatatccagttgatgaccaccacttcaaggtgattctgcactatgggaccctggttattgacggtgtcacgcccaacatgatcgactactttgggcggccctacgaggggattgccgtgtttgatggaaagaaaataacagtgaccgggactttatggaatggcaacaaaattattgatgagcgactgatcaaccctgatggatctctacttttccgtgtaactattaatggagtgactggctggagactgtgtgagagaatccttgcatgataggggggcggaggctcggtattcaccttggaagatttcgtgggtgattggaggcaaaccgctggctacaacctggaccaagtgctggagcaagggggcgtttcaagtctttttcaaaacctcggtgtgtccgtgacacccattcagaggatcgtactgtcgggggagaatgggctgaagatagatattcatgtgataatcccctatgaaggactcagtggggaccagatgggccaaattgaaaaaatcttcaaagtggtatatccagttgatgaccaccacttcaaggtgattctgcactatgggaccctggttattgacggtgtcacgcccaacatgatcgactactttgggcggccctacgaggggattgccgtgtttgatggaaagaaaataacagtgaccgggactttatggaatggcaacaaaattattgatgagcgactgatcaaccctgatggatctctacttttccgtgtaactattaatggagtgactggctggagactgtgtgagagaatccttgcagctcgctttcttgctgtccaatttctattaaaggttcctttgttccctaagtccaactactaaactgggggatattatgaagggccttgagcatctggattctgcctaataaaaaacatttattttcattgcgcggccgc

SEQ ID NO: 8 - DNA, 2874 bp, synthetic construct
  source 1..2874: mol_type = other DNA
atgaagtgggttaccttcatctctttgctcttcctgttttcgagtgcttattctcaagctgtggtcacccaggaatcagctctcacaacatcaccgggagagaccgtaactctaacatgtaggagctccacgggtgcagttaccacaagcaattatgccaactgggtccaggagaagcccgaccatctgttcacagggttgattggcggcaccaacaaccgagctcccggggtgcccgcacgcttctcagggtcactcattggtgataaagcagcattgaccatcacgggagcccagacggaagacgaggccatctacttctgtgccctgtggtacagcaacctctgggtgtttggtggtggtaccaagctgactgtactgagaagcgacgctgagccatctgttttcctcttcaaaccctccgatgagcaactgaaaactggcactgtctcagtagtgtgcctggtgaatgacttctacccgaaggatatcaatgtcaaatggaaggttgatggtgtgacacagtcctcttccaatttccagaacagttttaccgaccaggacagcaagaagtccacctattcgctaagcagcatactgacgctgcctagttccgagtaccagagtcatgatgcctacacatgtgaggtcagccacaagtctttgacgaccactctcgtgaagagtttcagcaaaaatgaatgtggaggtggcggcagtggggggggaggctctggagggggcggctccggcgggggaggcagtggaggggggggctccggaggtgggggttctggcggcggtggctcaggggggggaggatctggtggagggggatctggagggggtggatcaggtggtggcggctcgggaggcgggggtagtcaagtgcagctgaagcaggccggccccggcctggtacagcctagccagtctcttagcatcacgtgcacagtgagcggtttctcgttgtccaactatggagtgcactgggttaggcaaagtccaggcaagggcctggagtggttggggcttatttgggggggaggacgttcggattacaatgctgccttcatatcacgcctcaccatttcgaaggacaattctaaatctcaggtgttcttcactatgaactccctccaggtcaatgacactggtatttattactgcgtcaggaacggaggagaccccgcctggtttgcgtactggggccaaggaaccttagtgacagttagtgcggcctcaacaagcgccccgaaagtttaccctctgactagctgctgtggcgacaagagctcctcacgagtcactctgggctgccttgtcagttcctacatgccagagcctgtgacagtcacctggaactctggggcgttgaaaagcggagtccacacgtttccagcagtgctgcagtcctccggtctttattctctgagcagcatggtcactgtgcccggaagcacttcggggcagacctttacttgtaatgtggcccaccctgcctcctcaacaaaagtggacaaggccgtcggcgtatccagtgactgcagcaagccaaataaccagcattgtgtaagagaaccgagcgtattcatatttccccctaaaccaaaagacaccctgatgattactgggacaccagaagtcacctgcgtggtggtgaacgtcgggcatgacaaccccgaagttcagttctcctggttcgtggacgatgtggaggtgcataccgcgcggaccaagccaagagaggaacagtttaatagtacgtacagggttgtctccgccctgcccatccagcaccaggattggaccggggggaaagagtttaagtgcaaggttaatattaaaggtctctcagcttccattgtgcgcatcatcagcagatccaaggggccggcccgggagccccaggtgtatgtgcttgacccacccaaggaagagctgagcaaatcaacggtgtctgttacatgcatggtgatcggcttctatcctgaggatgtggacgtggaatggcagagggacagacaaacggagtctgaagataagtaccgcaccacccctcctcagttggatgctgataggtcctattttctctactctaagctccgcgtggatagaaacagctggcagcggggcggcacttacacctgtgtggtcatgcatgaagcactacacaaccactacatgcagaagtctacgtccaaaagtgcaggaaaaggaggcggtgggtcagtttttactctggaggattttgtgggagactggcgccaaacagctggctacaacctggaccaagtattagaacaaggaggtgtgagctccctctttcagaatctgggggtgagcgtaacacctattcaaagaatcgtcctgtcaggggagaatggactaaagattgacatccatgttatcattccatatgagggcttatccggggatcagatgggccagatcgagaagatatttaaagtggtgtacccagttgatgaccaccacttcaaggtgatcctgcactatgggaccctcgtgatagacggcgtcacaccgaacatgatcgactactttgggcggccctatgaaggcattgcagtctttgatggaaaaaagatcaccgtgactggcactttatggaatggaaataaaattatagatgaacggctgatcaatcctgatggatcgctacttttccgtgtgaccataaacggcgtcacaggctggaggctgtgtgaacgaatccttgcgggcggtggtggaagccccaacaaagggagcggcacaacttctgggaccactcggctcttgagtggccacacttgcttcacccttaccgggctcttaggaacgctggtcaccatgggactgctgacgtgatag

SEQ ID NO: 9 - DNA, 15 bp, synthetic construct
  source 1..15: mol_type = other DNA
ggaggcggtgggtca

SEQ ID NO: 10 - DNA, 510 bp, synthetic construct
  source 1..510: mol_type = other DNA
gtttttactctggaggattttgtgggagactggcgccaaacagctggctacaacctggaccaagtattagaacaaggaggtgtgagctccctctttcagaatctgggggtgagcgtaacacctattcaaagaatcgtcctgtcaggggagaatggactaaagattgacatccatgttatcattccatatgagggcttatccggggatcagatgggccagatcgagaagatatttaaagtggtgtacccagttgatgaccaccacttcaaggtgatcctgcactatgggaccctcgtgatagacggcgtcacaccgaacatgatcgactactttgggcggccctatgaaggcattgcagtctttgatggaaaaaagatcaccgtgactggcactttatggaatggaaataaaattatagatgaacggctgatcaatcctgatggatcgctacttttccgtgtgaccataaacggcgtcacaggctggaggctgtgtgaacgaatccttgcg

SEQ ID NO: 11 - DNA, 15 bp, synthetic construct
  source 1..15: mol_type = other DNA
ggcggtggtggaagc

SEQ ID NO: 12 - DNA, 111 bp, synthetic construct
  source 1..111: mol_type = other DNA
cccaacaaagggagcggcacaacttctgggaccactcggctcttgagtggccacacttgcttcacccttaccgggctcttaggaacgctggtcaccatgggactgctgacg

SEQ ID NO: 13 - DNA, 3735 bp, synthetic construct
  source 1..3735: mol_type = other DNA
aagctttaatacgactcactatagggaaataagagagaaaagaagagtaagaagaaatataagagccaccatgaagtgggttaccttcatctctttgctcttcctgttttcgagtgcttattctcaagctgtggtcacccaggaatcagctctcacaacatcaccgggagagaccgtaactctaacatgtaggagctccacgggtgcagttaccacaagcaattatgccaactgggtccaggagaagcccgaccatctgttcacagggttgattggcggcaccaacaaccgagctcccggggtgcccgcacgcttctcagggtcactcattggtgataaagcagcattgaccatcacgggagcccagacggaagacgaggccatctacttctgtgccctgtggtacagcaacctctgggtgtttggtggtggtaccaagctgactgtactgagaagcgacgctgagccatctgttttcctcttcaaaccctccgatgagcaactgaaaactggcactgtctcagtagtgtgcctggtgaatgacttctacccgaaggatatcaatgtcaaatggaaggttgatggtgtgacacagtcctcttccaatttccagaacagttttaccgaccaggacagcaagaagtccacctattcgctaagcagcatactgacgctgcctagttccgagtaccagagtcatgatgcctacacatgtgaggtcagccacaagtctttgacgaccactctcgtgaagagtttcagcaaaaatgaatgtggaggtggcggcagtggggggggaggctctggagggggcggctccggcgggggaggcagtggaggggggggctccggaggtgggggttctggcggcggtggctcaggggggggaggatctggtggagggggatctggagggggtggatcaggtggtggcggctcgggaggcgggggtagtcaagtgcagctgaagcaggccggccccggcctggtacagcctagccagtctcttagcatcacgtgcacagtgagcggtttctcgttgtccaactatggagtgcactgggttaggcaaagtccaggcaagggcctggagtggttggggcttatttgggggggaggacgttcggattacaatgctgccttcatatcacgcctcaccatttcgaaggacaattctaaatctcaggtgttcttcactatgaactccctccaggtcaatgacactggtatttattactgcgtcaggaacggaggagaccccgcctggtttgcgtactggggccaaggaaccttagtgacagttagtgcggcctcaacaagcgccccgaaagtttaccctctgactagctgctgtggcgacaagagctcctcacgagtcactctgggctgccttgtcagttcctacatgccagagcctgtgacagtcacctggaactctggggcgttgaaaagcggagtccacacgtttccagcagtgctgcagtcctccggtctttattctctgagcagcatggtcactgtgcccggaagcacttcggggcagacctttacttgtaatgtggcccaccctgcctcctcaacaaaagtggacaaggccgtcggcgtatccagtgactgcagcaagccaaataaccagcattgtgtaagagaaccgagcgtattcatatttccccctaaaccaaaagacaccctgatgattactgggacaccagaagtcacctgcgtggtggtgaacgtcgggcatgacaaccccgaagttcagttctcctggttcgtggacgatgtggaggtgcataccgcgcggaccaagccaagagaggaacagtttaatagtacgtacagggttgtctccgccctgcccatccagcaccaggattggaccggggggaaagagtttaagtgcaaggttaatattaaaggtctctcagcttccattgtgcgcatcatcagcagatccaaggggccggcccgggagccccaggtgtatgtgcttgacccacccaaggaagagctgagcaaatcaacggtgtctgttacatgcatggtgatcggcttctatcctgaggatgtggacgtggaatggcagagggacagacaaacggagtctgaagataagtaccgcaccacccctcctcagttggatgctgataggtcctattttctctactctaagctccgcgtggatagaaacagctggcagcggggcggcacttacacctgtgtggtcatgcatgaagcactacacaaccactacatgcagaagtctacgtccaaaagtgcaggaaaaggaggcggtgggtcagtttttactctggaggattttgtgggagactggcgccaaacagctggctacaacctggaccaagtattagaacaaggaggtgtgagctccctctttcagaatctgggggtgagcgtaacacctattcaaagaatcgtcctgtcaggggagaatggactaaagattgacatccatgttatcattccatatgagggcttatccggggatcagatgggccagatcgagaagatatttaaagtggtgtacccagttgatgaccaccacttcaaggtgatcctgcactatgggaccctcgtgatagacggcgtcacaccgaacatgatcgactactttgggcggccctatgaaggcattgcagtctttgatggaaaaaagatcaccgtgactggcactttatggaatggaaataaaattatagatgaacggctgatcaatcctgatggatcgctacttttccgtgtgaccataaacggcgtcacaggctggaggctgtgtgaacgaatccttgcgggcggtggtggaagccccaacaaagggagcggcacaacttctgggaccactcggctcttgagtggccacacttgcttcacccttaccgggctcttaggaacgctggtcaccatgggactgctgacgtgatagggaggcggtgggtcagtttttactctggaggattttgtgggagactggcgccaaacagctggctacaacctggaccaagtattagaacaaggaggtgtgagctccctctttcagaatctgggggtgagcgtaacacctattcaaagaatcgtcctgtcaggggagaatggactaaagattgacatccatgttatcattccatatgagggcttatccggggatcagatgggccagatcgagaagatatttaaagtggtgtacccagttgatgaccaccacttcaaggtgatcctgcactatgggaccctcgtgatagacggcgtcacaccgaacatgatcgactactttgggcggccctatgaaggcattgcagtctttgatggaaaaaagatcaccgtgactggcactttatggaatggaaataaaattatagatgaacggctgatcaatcctgatggatcgctacttttccgtgtgaccataaacggcgtcacaggctggaggctgtgtgaacgaatccttgcgggcggtggtggaagccccaacaaagggagcggcacaacttctgggaccactcggctcttgagtggccacacttgcttcacccttaccgggctcttaggaacgctggtcaccatgggactgctgacggctcgctttcttgctgtccaatttctattaaaggttcctttgttccctaagtccaactactaaactgggggatattatgaagggccttgagcatctggattctgcctaataaaaaacatttattttcattgcgcggccgc

SEQ ID NO: 14 - DNA, 2754 bp, synthetic construct
  source 1..2754: mol_type = other DNA
atgaagtgggtgacctttatctcgttactcttcctattttcttcggcctactcagacatcgtgatgtctcagtctccgagttccctggcagttagcgtcggagaaaaagtgaccatgagctgtaagagttctcagtcactcctgtacagtagtaatcaaaaaaactacctggcctggtaccagcagaagccgggacagagccccaaacttttaatatactgggccagcactcgcgagtccggtgtcccggatcgcttcaccggatcaggatccggtacagattttaccttaactatctcctccgtgaaggcagaggatttagcagtctattactgccagcagtactataattatccattcacgtttggaggaggtaccaacctggagatcaagaggagtgacgccgagccaagcgtctttctgtttaaaccttcagatgagcaactaaagaccgggacggtatcagtggtgtgcctggtgaatgacttctatcctaaagacatcaatgtgaaatggaaggttgacggggtcactcagtccagctcaaattttcagaattcattcactgaccaggactcaaagaagagtacttattccttgagttccatccttacacttccctcttcggaatatcagagccatgacgcgtatacctgtgaggtgagccacaaatctcttacaacgactttggtgaaaagcttttcaaagaatgaatgtggcggtggcggttcaggggggggcggctctggcggcggtggcagtggcggcggaggttccgggggaggcggatccggaggagggggcagcggtggcggcggaagtggtggcgggggctccgggggaggaggatcaggggggggtgggagtggtggcgggggctcagggggaggaggttcccaagttcagctgaagcagagtggacccggactcgtacagcccagccaaaacttgtccattacatgtacagtctctggcttcagcctgacgacctatggcgtgcactgggtcaggcaaagtcctggcaagggcctggactggctcggggtcatttggggggggggctccacagacttcaatgcagcctttatgtcgaggctctccatcactaaagataacagcaaatcccaggtcttcttcaagatgaactccctgcaggccgatgatacagccatctactactgtgccaagagccttctatcaagcatggattactggggccaaggcacgtccgtcaccgtcagttcagcgtctacgtcggctcctaaagtctatccgctcacttcgtgctgcggggacaaatcgtcatctagggttacactgggctgcctagtttctagctacatgcccgaacccgttacagttacgtggaatagcggggctcttaagtctggtgtgcacaccttccccgcagtgttgcagtcatccggtctctactccctgagctccatggtgacggtgcccgggagcaccagcggccaaacatttacttgcaatgtagcgcatccagcatcgtcgacaaaagtagacaaagctgtgggagttagctcggactgcagcaagcccaacaaccagcactgcgtaagagagccctctgtctttatcttcccaccaaaaccaaaggacaccctcatgattaccgggactcccgaagtaacctgtgtagttgtgaatgttggccacgacaatccagaggtgcagttctcctggttcgtggatgacgtggaagttcatactgctcgcaccaagcctcgggaggaacagtttaacagtacctacagagtcgtgtcagccctgcctatccagcatcaggattggacaggggggaaagagttcaagtgcaaagtgaacatcaaagggctcagcgccagcattgtccgtataatttccaggtctaagggccctgctcgagaaccccaggtatacgtgctggatccacccaaggaggagctctccaagtcaaccgtttccgtgacctgcatggtgattggattctaccctgaggacgtcgatgtggagtggcagagagaccggcagacggagagtgaagacaagtacagaaccaccccaccccaactggatgctgaccgatcctacttcctgtatagcaagctgcgggtggataggaatagctggcagcggggaggcacatatacatgtgtggtcatgcatgaagcacttcacaaccactacatgcagaaatctacatctaagtctgctgggaagggtggcggaggcagcgtttttacactggaagattttgtgggcgactggagacagactgccggctacaacttggaccaagtgctcgagcagggcggggtcagctccttgttccagaaccttggggttagcgtgactcctattcaacgaattgttctgagtggcgaaaacggattaaagattgatattcatgtgatcatcccgtatgaggggctgtcgggggaccagatgggccagatcgagaaaatctttaaagtggtgtaccctgtcgacgatcaccacttcaaagtcatcctgcactatggaaccctggtgatagatggtgtaacgccaaacatgatagactattttggtcggccttacgagggcatagccgtgtttgatggcaaaaagataacagtaactggtaccctgtggaacggcaacaagattattgacgaacgtctgatcaaccctgatggatcgctactgttccgggtgaccattaatggagtcactggctggcgcctgtgtgaaagaatcctggcttgatag

SEQ ID NO: 15 - DNA, 15 bp, synthetic construct
  source 1..15: mol_type = other DNA
ggtggcggaggcagc

SEQ ID NO: 16 - DNA, 510 bp, synthetic construct
  source 1..510: mol_type = other DNA
gtttttacactggaagattttgtgggcgactggagacagactgccggctacaacttggaccaagtgctcgagcagggcggggtcagctccttgttccagaaccttggggttagcgtgactcctattcaacgaattgttctgagtggcgaaaacggattaaagattgatattcatgtgatcatcccgtatgaggggctgtcgggggaccagatgggccagatcgagaaaatctttaaagtggtgtaccctgtcgacgatcaccacttcaaagtcatcctgcactatggaaccctggtgatagatggtgtaacgccaaacatgatagactattttggtcggccttacgagggcatagccgtgtttgatggcaaaaagataacagtaactggtaccctgtggaacggcaacaagattattgacgaacgtctgatcaaccctgatggatcgctactgttccgggtgaccattaatggagtcactggctggcgcctgtgtgaaagaatcctggct

SEQ ID NO: 17 - DNA, 3489 bp, synthetic construct
  source 1..3489: mol_type = other DNA
aagctttaatacgactcactatagggaaataagagagaaaagaagagtaagaagaaatataagagccaccatgaagtgggtgacctttatctcgttactcttcctattttcttcggcctactcagacatcgtgatgtctcagtctccgagttccctggcagttagcgtcggagaaaaagtgaccatgagctgtaagagttctcagtcactcctgtacagtagtaatcaaaaaaactacctggcctggtaccagcagaagccgggacagagccccaaacttttaatatactgggccagcactcgcgagtccggtgtcccggatcgcttcaccggatcaggatccggtacagattttaccttaactatctcctccgtgaaggcagaggatttagcagtctattactgccagcagtactataattatccattcacgtttggaggaggtaccaacctggagatcaagaggagtgacgccgagccaagcgtctttctgtttaaaccttcagatgagcaactaaagaccgggacggtatcagtggtgtgcctggtgaatgacttctatcctaaagacatcaatgtgaaatggaaggttgacggggtcactcagtccagctcaaattttcagaattcattcactgaccaggactcaaagaagagtacttattccttgagttccatccttacacttccctcttcggaatatcagagccatgacgcgtatacctgtgaggtgagccacaaatctcttacaacgactttggtgaaaagcttttcaaagaatgaatgtggcggtggcggttcaggggggggcggctctggcggcggtggcagtggcggcggaggttccgggggaggcggatccggaggagggggcagcggtggcggcggaagtggtggcgggggctccgggggaggaggatcaggggggggtgggagtggtggcgggggctcagggggaggaggttcccaagttcagctgaagcagagtggacccggactcgtacagcccagccaaaacttgtccattacatgtacagtctctggcttcagcctgacgacctatggcgtgcactgggtcaggcaaagtcctggcaagggcctggactggctcggggtcatttggggggggggctccacagacttcaatgcagcctttatgtcgaggctctccatcactaaagataacagcaaatcccaggtcttcttcaagatgaactccctgcaggccgatgatacagccatctactactgtgccaagagccttctatcaagcatggattactggggccaaggcacgtccgtcaccgtcagttcagcgtctacgtcggctcctaaagtctatccgctcacttcgtgctgcggggacaaatcgtcatctagggttacactgggctgcctagtttctagctacatgcccgaacccgttacagttacgtggaatagcggggctcttaagtctggtgtgcacaccttccccgcagtgttgcagtcatccggtctctactccctgagctccatggtgacggtgcccgggagcaccagcggccaaacatttacttgcaatgtagcgcatccagcatcgtcgacaaaagtagacaaagctgtgggagttagctcggactgcagcaagcccaacaaccagcactgcgtaagagagccctctgtctttatcttcccaccaaaaccaaaggacaccctcatgattaccgggactcccgaagtaacctgtgtagttgtgaatgttggccacgacaatccagaggtgcagttctcctggttcgtggatgacgtggaagttcatactgctcgcaccaagcctcgggaggaacagtttaacagtacctacagagtcgtgtcagccctgcctatccagcatcaggattggacaggggggaaagagttcaagtgcaaagtgaacatcaaagggctcagcgccagcattgtccgtataatttccaggtctaagggccctgctcgagaaccccaggtatacgtgctggatccacccaaggaggagctctccaagtcaaccgtttccgtgacctgcatggtgattggattctaccctgaggacgtcgatgtggagtggcagagagaccggcagacggagagtgaagacaagtacagaaccaccccaccccaactggatgctgaccgatcctacttcctgtatagcaagctgcgggtggataggaatagctggcagcggggaggcacatatacatgtgtggtcatgcatgaagcacttcacaaccactacatgcagaaatctacatctaagtctgctgggaagggtggcggaggcagcgtttttacactggaagattttgtgggcgactggagacagactgccggctacaacttggaccaagtgctcgagcagggcggggtcagctccttgttccagaaccttggggttagcgtgactcctattcaacgaattgttctgagtggcgaaaacggattaaagattgatattcatgtgatcatcccgtatgaggggctgtcgggggaccagatgggccagatcgagaaaatctttaaagtggtgtaccctgtcgacgatcaccacttcaaagtcatcctgcactatggaaccctggtgatagatggtgtaacgccaaacatgatagactattttggtcggccttacgagggcatagccgtgtttgatggcaaaaagataacagtaactggtaccctgtggaacggcaacaagattattgacgaacgtctgatcaaccctgatggatcgctactgttccgggtgaccattaatggagtcactggctggcgcctgtgtgaaagaatcctggcttgatagggtggcggaggcagcgtttttacactggaagattttgtgggcgactggagacagactgccggctacaacttggaccaagtgctcgagcagggcggggtcagctccttgttccagaaccttggggttagcgtgactcctattcaacgaattgttctgagtggcgaaaacggattaaagattgatattcatgtgatcatcccgtatgaggggctgtcgggggaccagatgggccagatcgagaaaatctttaaagtggtgtaccctgtcgacgatcaccacttcaaagtcatcctgcactatggaaccctggtgatagatggtgtaacgccaaacatgatagactattttggtcggccttacgagggcatagccgtgtttgatggcaaaaagataacagtaactggtaccctgtggaacggcaacaagattattgacgaacgtctgatcaaccctgatggatcgctactgttccgggtgaccattaatggagtcactggctggcgcctgtgtgaaagaatcctggctgctcgctttcttgctgtccaatttctattaaaggttcctttgttccctaagtccaactactaaactgggggatattatgaagggccttgagcatctggattctgcctaataaaaaacatttattttcattgcgcggccgc

SEQ ID NO: 18 - DNA, 2880 bp, synthetic construct
  source 1..2880: mol_type = other DNA
atgaaatgggtcacgttcatcagtctgctatttttgttctcctccgcttacagcgacattgtgatgagtcaaagtccctcttctctggcggtgagtgtcggagagaaagtgacaatgtcttgcaagagtagccagtcattgctgtacagcagtaaccagaagaactatctcgcttggtaccagcagaaaccaggtcagtcacctaaactgctgatatactgggcatcaacccgagaatcaggcgtaccagaccggtttactgggtccgggagcggtacggacttcacgcttacaatatcatccgtaaaagcagaggaccttgcagtttactactgccagcagtactataattatcccttcacattcggcggaggcaccaacctggaaatcaagagatcagatgctgaaccatctgtgtttctttttaagccctccgacgaacagctgaagacaggtactgtcagtgtggtatgcttagttaacgacttctacccgaaggatattaatgtcaagtggaaggtggacggcgtaactcaaagttcaagcaacttccaaaattcatttacggaccaagatagcaagaaatcaacttattccctttcatcgattttaactctcccctcgtctgagtatcaatcgcatgacgcctacacttgtgaggtttctcacaagtccttgacaaccacattggtgaaatcctttagcaagaatgaatgtggcgggggcggaagcggcggcggcggttctggtggaggaggcagcggaggaggcggctccgggggagggggaagcggcggtggagggtccggaggaggcggctcaggcgggggggggtcgggaggggggggaagcggagggggtggaagcggaggcgggggatctggaggcggcggctctcaggttcaactcaagcagtccggtcccggactggttcagccatcacagaacctctctataacctgcactgtctccggctttagtctgaccacctatggtgtgcactgggtgcggcagtctcctggcaaggggctggattggctgggtgttatctggggtgggggaagtaccgattttaatgcagccttcatgagccgcctatctattaccaaggacaattcgaagagccaggtgttcttcaagatgaactctctgcaggccgacgacaccgcaatttattactgtgccaagtctctgctgtcttcgatggactactggggccaagggacgtctgtcaccgtgagttccgctagtacgtccgcgcccaaggtgtaccccctcaccagctgttgtggtgataaaagcagctctcgagttacactcggctgcctcgtgtccagctatatgcctgaacccgtcaccgtcacttggaacagtggcgcactgaaaagcggcgtgcacacatttccggcggtcctccagagtagcgggctctactccctctctagcatggtgactgtgcctgggagcacaagtggccagacctttacctgcaatgtggcccatccagcctccagcaccaaagtcgataaggctgtgggagtttccagtgactgcagcaagcctaataatcagcattgcgtgagggaacctagcgtttttatttttcccccaaagccgaaagataccctgatgataacaggaaccccggaggtaacttgtgtggtggtgaatgtggggcatgacaacccagaagtgcagttctcctggttcgtggatgatgtagaggtccatactgcccgtacaaaaccccgggaggagcagtttaattcgacgtacagagtagtttcagcacttcccatccagcaccaggactggacaggcggaaaggaatttaagtgcaaagtgaacatcaaaggtttgtcagcctccatcgtacgcatcatttctcgtagtaagggccctgcccgagagcctcaagtctatgtactggacccacctaaagaagagctgtccaaatctacggtgagcgtgacgtgcatggtcattggcttctatccagaagatgttgacgtggagtggcagagggacaggcaaactgagagcgaggacaaataccggaccacacctccgcagcttgatgctgacagaagctatttcctgtactccaagctaagggtggacagaaactcctggcagcgggggggtacttacacctgtgtagtcatgcatgaggccctgcacaaccactacatgcagaaatcaacatccaagagtgccggcaaagggggcggcggctctgttttcactttggaagacttcgttggagattggagacaaacagctggctacaacttggatcaggtgttggagcagggcggtgtgtctagcctcttccagaatctgggagtgagcgttactccaattcagcgcatcgtcttaagcggggagaacggcctcaaaatcgacattcacgtgatcatcccctacgagggactttcgggggatcagatggggcagatcgagaagatcttcaaagtcgtttatcctgtggatgaccaccactttaaggtcatcctacattatggaactttagtcatagatggcgtgacacccaacatgatcgactattttgggaggccttatgaaggcatcgctgtctttgatggaaagaagattacagttacaggcaccctgtggaatgggaacaaaataatagatgagcggctgattaaccctgatggctccttactgttccgcgtcacaatcaatggagtaaccggctggaggctctgtgaacgcattctggccggcgggggtgggagcccaaacaaaggcagtggtaccacatcaggcaccacgagacttctctctggacacacctgtttcacactgaccggcctgcttggaacactcgtcactatgggtcttctgacgtgatag

SEQ ID NO: 19 - DNA, 15 bp, synthetic construct
  source 1..15: mol_type = other DNA
gggggcggcggctct

SEQ ID NO: 20 - DNA, 510 bp, synthetic construct
  source 1..510: mol_type = other DNA
gttttcactttggaagacttcgttggagattggagacaaacagctggctacaacttggatcaggtgttggagcagggcggtgtgtctagcctcttccagaatctgggagtgagcgttactccaattcagcgcatcgtcttaagcggggagaacggcctcaaaatcgacattcacgtgatcatcccctacgagggactttcgggggatcagatggggcagatcgagaagatcttcaaagtcgtttatcctgtggatgaccaccactttaaggtcatcctacattatggaactttagtcatagatggcgtgacacccaacatgatcgactattttgggaggccttatgaaggcatcgctgtctttgatggaaagaagattacagttacaggcaccctgtggaatgggaacaaaataatagatgagcggctgattaaccctgatggctccttactgttccgcgtcacaatcaatggagtaaccggctggaggctctgtgaacgcattctggcc

SEQ ID NO: 21 - DNA, 15 bp, synthetic construct
  source 1..15: mol_type = other DNA
ggcgggggtgggagc

SEQ ID NO: 22 - DNA, 111 bp, synthetic construct
  source 1..111: mol_type = other DNA
ccaaacaaaggcagtggtaccacatcaggcaccacgagacttctctctggacacacctgtttcacactgaccggcctgcttggaacactcgtcactatgggtcttctgacg

SEQ ID NO: 23 - DNA, 3741 bp, synthetic construct
  source 1..3741: mol_type = other DNA
aagctttaatacgactcactatagggaaataagagagaaaagaagagtaagaagaaatataagagccaccatgaaatgggtcacgttcatcagtctgctatttttgttctcctccgcttacagcgacattgtgatgagtcaaagtccctcttctctggcggtgagtgtcggagagaaagtgacaatgtcttgcaagagtagccagtcattgctgtacagcagtaaccagaagaactatctcgcttggtaccagcagaaaccaggtcagtcacctaaactgctgatatactgggcatcaacccgagaatcaggcgtaccagaccggtttactgggtccgggagcggtacggacttcacgcttacaatatcatccgtaaaagcagaggaccttgcagtttactactgccagcagtactataattatcccttcacattcggcggaggcaccaacctggaaatcaagagatcagatgctgaaccatctgtgtttctttttaagccctccgacgaacagctgaagacaggtactgtcagtgtggtatgcttagttaacgacttctacccgaaggatattaatgtcaagtggaaggtggacggcgtaactcaaagttcaagcaacttccaaaattcatttacggaccaagatagcaagaaatcaacttattccctttcatcgattttaactctcccctcgtctgagtatcaatcgcatgacgcctacacttgtgaggtttctcacaagtccttgacaaccacattggtgaaatcctttagcaagaatgaatgtggcgggggcggaagcggcggcggcggttctggtggaggaggcagcggaggaggcggctccgggggagggggaagcggcggtggagggtccggaggaggcggctcaggcgggggggggtcgggaggggggggaagcggagggggtggaagcggaggcgggggatctggaggcggcggctctcaggttcaactcaagcagtccggtcccggactggttcagccatcacagaacctctctataacctgcactgtctccggctttagtctgaccacctatggtgtgcactgggtgcggcagtctcctggcaaggggctggattggctgggtgttatctggggtgggggaagtaccgattttaatgcagccttcatgagccgcctatctattaccaaggacaattcgaagagccaggtgttcttcaagatgaactctctgcaggccgacgacaccgcaatttattactgtgccaagtctctgctgtcttcgatggactactggggccaagggacgtctgtcaccgtgagttccgctagtacgtccgcgcccaaggtgtaccccctcaccagctgttgtggtgataaaagcagctctcgagttacactcggctgcctcgtgtccagctatatgcctgaacccgtcaccgtcacttggaacagtggcgcactgaaaagcggcgtgcacacatttccggcggtcctccagagtagcgggctctactccctctctagcatggtgactgtgcctgggagcacaagtggccagacctttacctgcaatgtggcccatccagcctccagcaccaaagtcgataaggctgtgggagtttccagtgactgcagcaagcctaataatcagcattgcgtgagggaacctagcgtttttatttttcccccaaagccgaaagataccctgatgataacaggaaccccggaggtaacttgtgtggtggtgaatgtggggcatgacaacccagaagtgcagttctcctggttcgtggatgatgtagaggtccatactgcccgtacaaaaccccgggaggagcagtttaattcgacgtacagagtagtttcagcacttcccatccagcaccaggactggacaggcggaaaggaatttaagtgcaaagtgaacatcaaaggtttgtcagcctccatcgtacgcatcatttctcgtagtaagggccctgcccgagagcctcaagtctatgtactggacccacctaaagaagagctgtccaaatctacggtgagcgtgacgtgcatggtcattggcttctatccagaagatgttgacgtggagtggcagagggacaggcaaactgagagcgaggacaaataccggaccacacctccgcagcttgatgctgacagaagctatttcctgtactccaagctaagggtggacagaaactcctggcagcgggggggtacttacacctgtgtagtcatgcatgaggccctgcacaaccactacatgcagaaatcaacatccaagagtgccggcaaagggggcggcggctctgttttcactttggaagacttcgttggagattggagacaaacagctggctacaacttggatcaggtgttggagcagggcggtgtgtctagcctcttccagaatctgggagtgagcgttactccaattcagcgcatcgtcttaagcggggagaacggcctcaaaatcgacattcacgtgatcatcccctacgagggactttcgggggatcagatggggcagatcgagaagatcttcaaagtcgtttatcctgtggatgaccaccactttaaggtcatcctacattatggaactttagtcatagatggcgtgacacccaacatgatcgactattttgggaggccttatgaaggcatcgctgtctttgatggaaagaagattacagttacaggcaccctgtggaatgggaacaaaataatagatgagcggctgattaaccctgatggctccttactgttccgcgtcacaatcaatggagtaaccggctggaggctctgtgaacgcattctggccggcgggggtgggagcccaaacaaaggcagtggtaccacatcaggcaccacgagacttctctctggacacacctgtttcacactgaccggcctgcttggaacactcgtcactatgggtcttctgacgtgataggggggcggcggctctgttttcactttggaagacttcgttggagattggagacaaacagctggctacaacttggatcaggtgttggagcagggcggtgtgtctagcctcttccagaatctgggagtgagcgttactccaattcagcgcatcgtcttaagcggggagaacggcctcaaaatcgacattcacgtgatcatcccctacgagggactttcgggggatcagatggggcagatcgagaagatcttcaaagtcgtttatcctgtggatgaccaccactttaaggtcatcctacattatggaactttagtcatagatggcgtgacacccaacatgatcgactattttgggaggccttatgaaggcatcgctgtctttgatggaaagaagattacagttacaggcaccctgtggaatgggaacaaaataatagatgagcggctgattaaccctgatggctccttactgttccgcgtcacaatcaatggagtaaccggctggaggctctgtgaacgcattctggccggcgggggtgggagcccaaacaaaggcagtggtaccacatcaggcaccacgagacttctctctggacacacctgtttcacactgaccggcctgcttggaacactcgtcactatgggtcttctgacggctcgctttcttgctgtccaatttctattaaaggttcctttgttccctaagtccaactactaaactgggggatattatgaagggccttgagcatctggattctgcctaataaaaaacatttattttcattgcgcggccgc

SEQ ID NO: 24 - DNA, 1395 bp, synthetic construct
  source 1..1395: mol_type = other DNA
atggccgtgctgggcctgctgttctgcctggtgaccttcccttcctgcgtgctgagccaggtgcagctgaagcaggctggacctggactggtgcagccttcccagagcctgtccatcacctgcaccgtgtccggcttcagcctgtccaactacggagtgcactgggtgcggcagagccctggcaagggcctggagtggctgggactgatctggggaggcggccggtccgactacaacgctgccttcatctccaggctgaccatcagcaaggacaacagcaagtcccaggtgttcttcaccatgaacagcctgcaggtgaacgacaccggcatctactactgcgtgcggaacggaggcgaccctgcttggttcgcttactggggacagggcaccctggtgaccgtgtccgccgctagcacctccgcccccaaggtgtaccctctgaccagctgctgcggcgacaagagctccagcagggtgaccctgggatgcctggtgtccagctacatgcccgagcctgtgaccgtgacctggaactccggcgccctgaagagcggagtgcacaccttccctgccgtgctgcagtccagcggactgtactccctgtccagcatggtgaccgtgcctggaagcacctccggacagaccttcacctgcaacgtggctcaccctgcttccagcaccaaggtggacaaggccgtgggcgtgtccagcgactgctccaagcccaacaaccagcactgcgtgcgggagcctagcgtgttcatcttcccccctaagcccaaggacaccctgatgatcaccggaacccctgaggtgacctgcgtggtggtgaacgtgggccacgacaaccccgaggtgcagttcagctggttcgtggacgacgtggaggtgcacaccgctaggaccaagcctagggaggagcagttcaactccacctacagggtggtgagcgccctgcctatccagcaccaggactggaccggaggcaaggagttcaagtgcaaggtgaacatcaagggcctgagcgcctccatcgtgcgcatcatcagcagatccaagggccccgccagagagcctcaggtgtacgtgctggacccccctaaggaggagctgagcaagtccaccgtgtccgtgacctgcatggtcatcggcttctaccctgaggacgtggacgtggagtggcagagggacaggcagaccgagagcgaggacaagtacaggaccacccctcctcagctggacgctgacagatcctacttcctgtacagcaagctgcgcgtggacagaaacagctggcagaggggaggaacctacacctgcgtggtcatgcacgaggccctgcacaaccactacatgcagaagagcacctccaagagcgccggcaagtgatga

SEQ ID NO: 25 - DNA, 93 bp, synthetic construct
  source 1..93: mol_type = other DNA
gctgccttctgcggggcttgccttctggccatgcccttcttctctcccttgcacctgtacctcttggtctttgaataaagcctgagtaggaag

SEQ ID NO: 26 - DNA, 1566 bp, synthetic construct
  source 1..1566: mol_type = other DNA
aagctttaatacgactcactatagggaaataagagagaaaagaagagtaagaagaaatataagagccaccatggccgtgctgggcctgctgttctgcctggtgaccttcccttcctgcgtgctgagccaggtgcagctgaagcaggctggacctggactggtgcagccttcccagagcctgtccatcacctgcaccgtgtccggcttcagcctgtccaactacggagtgcactgggtgcggcagagccctggcaagggcctggagtggctgggactgatctggggaggcggccggtccgactacaacgctgccttcatctccaggctgaccatcagcaaggacaacagcaagtcccaggtgttcttcaccatgaacagcctgcaggtgaacgacaccggcatctactactgcgtgcggaacggaggcgaccctgcttggttcgcttactggggacagggcaccctggtgaccgtgtccgccgctagcacctccgcccccaaggtgtaccctctgaccagctgctgcggcgacaagagctccagcagggtgaccctgggatgcctggtgtccagctacatgcccgagcctgtgaccgtgacctggaactccggcgccctgaagagcggagtgcacaccttccctgccgtgctgcagtccagcggactgtactccctgtccagcatggtgaccgtgcctggaagcacctccggacagaccttcacctgcaacgtggctcaccctgcttccagcaccaaggtggacaaggccgtgggcgtgtccagcgactgctccaagcccaacaaccagcactgcgtgcgggagcctagcgtgttcatcttcccccctaagcccaaggacaccctgatgatcaccggaacccctgaggtgacctgcgtggtggtgaacgtgggccacgacaaccccgaggtgcagttcagctggttcgtggacgacgtggaggtgcacaccgctaggaccaagcctagggaggagcagttcaactccacctacagggtggtgagcgccctgcctatccagcaccaggactggaccggaggcaaggagttcaagtgcaaggtgaacatcaagggcctgagcgcctccatcgtgcgcatcatcagcagatccaagggccccgccagagagcctcaggtgtacgtgctggacccccctaaggaggagctgagcaagtccaccgtgtccgtgacctgcatggtcatcggcttctaccctgaggacgtggacgtggagtggcagagggacaggcagaccgagagcgaggacaagtacaggaccacccctcctcagctggacgctgacagatcctacttcctgtacagcaagctgcgcgtggacagaaacagctggcagaggggaggaacctacacctgcgtggtcatgcacgaggccctgcacaaccactacatgcagaagagcacctccaagagcgccggcaagtgatgagctgccttctgcggggcttgccttctggccatgcccttcttctctcccttgcacctgtacctcttggtctttgaataaagcctgagtaggaaggcggccgc

SEQ ID NO: 27 - DNA, 714 bp, synthetic construct
  source 1..714: mol_type = other DNA
atggcctggatcagcctgatcctgtccctgctggccctgagctccggagccatcagccaggctgtggtgacccaggagagcgccctgaccacctcccctggagagaccgtgaccctgacctgccgcagctccaccggagctgtgaccacctccaactacgccaactgggtgcaggagaagcctgaccacctgttcaccggcctgatcggaggaaccaacaacagggctcctggagtgcctgccaggttctccggcagcctgatcggcgacaaggccgccctgaccatcaccggagctcagaccgaggacgaggccatctacttctgcgccctgtggtacagcaacctgtgggtgttcggaggaggaaccaagctgaccgtgctgcggagcgacgctgagccttccgtgttcctgttcaagcctagcgacgagcagctgaagaccggcaccgtgtccgtggtgtgcctggtgaacgacttctaccccaaggacatcaacgtgaagtggaaggtggacggcgtgacccagagctccagcaacttccagaacagcttcaccgaccaggactccaagaagagcacctactccctgtccagcatcctgaccctgccttccagcgagtaccagtcccacgacgcctacacctgcgaggtgagccacaagtccctgaccaccaccctggtgaagtccttcagcaagaacgagtgctgatga

SEQ ID NO: 28 - DNA, 885 bp, synthetic construct
  source 1..885: mol_type = other DNA
aagctttaatacgactcactatagggaaataagagagaaaagaagagtaagaagaaatataagagccaccatggcctggatcagcctgatcctgtccctgctggccctgagctccggagccatcagccaggctgtggtgacccaggagagcgccctgaccacctcccctggagagaccgtgaccctgacctgccgcagctccaccggagctgtgaccacctccaactacgccaactgggtgcaggagaagcctgaccacctgttcaccggcctgatcggaggaaccaacaacagggctcctggagtgcctgccaggttctccggcagcctgatcggcgacaaggccgccctgaccatcaccggagctcagaccgaggacgaggccatctacttctgcgccctgtggtacagcaacctgtgggtgttcggaggaggaaccaagctgaccgtgctgcggagcgacgctgagccttccgtgttcctgttcaagcctagcgacgagcagctgaagaccggcaccgtgtccgtggtgtgcctggtgaacgacttctaccccaaggacatcaacgtgaagtggaaggtggacggcgtgacccagagctccagcaacttccagaacagcttcaccgaccaggactccaagaagagcacctactccctgtccagcatcctgaccctgccttccagcgagtaccagtcccacgacgcctacacctgcgaggtgagccacaagtccctgaccaccaccctggtgaagtccttcagcaagaacgagtgctgatgagctgccttctgcggggcttgccttctggccatgcccttcttctctcccttgcacctgtacctcttggtctttgaataaagcctgagtaggaaggcggccgc

SEQ ID NO: 29 - DNA, 1518 bp, synthetic construct
  source 1..1518: mol_type = other DNA
atggccgtgctgggcctgctgttctgcctggtgaccttcccttcctgcgtgctgagccaggtgcagctgaagcaggctggacctggactggtgcagccttcccagagcctgtccatcacctgcaccgtgtccggcttcagcctgtccaactacggagtgcactgggtgcggcagagccctggcaagggcctggagtggctgggactgatctggggaggcggccggtccgactacaacgctgccttcatctccaggctgaccatcagcaaggacaacagcaagtcccaggtgttcttcaccatgaacagcctgcaggtgaacgacaccggcatctactactgcgtgcggaacggaggcgaccctgcttggttcgcttactggggacagggcaccctggtgaccgtgtccgccgctagcacctccgcccccaaggtgtaccctctgaccagctgctgcggcgacaagagctccagcagggtgaccctgggatgcctggtgtccagctacatgcccgagcctgtgaccgtgacctggaactccggcgccctgaagagcggagtgcacaccttccctgccgtgctgcagtccagcggactgtactccctgtccagcatggtgaccgtgcctggaagcacctccggacagaccttcacctgcaacgtggctcaccctgcttccagcaccaaggtggacaaggccgtgggcgtgtccagcgactgctccaagcccaacaaccagcactgcgtgcgggagcctagcgtgttcatcttcccccctaagcccaaggacaccctgatgatcaccggaacccctgaggtgacctgcgtggtggtgaacgtgggccacgacaaccccgaggtgcagttcagctggttcgtggacgacgtggaggtgcacaccgctaggaccaagcctagggaggagcagttcaactccacctacagggtggtgagcgccctgcctatccagcaccaggactggaccggaggcaaggagttcaagtgcaaggtgaacatcaagggcctgagcgcctccatcgtgcgcatcatcagcagatccaagggccccgccagagagcctcaggtgtacgtgctggacccccctaaggaggagctgagcaagtccaccgtgtccgtgacctgcatggtcatcggcttctaccctgaggacgtggacgtggagtggcagagggacaggcagaccgagagcgaggacaagtacaggaccacccctcctcagctggacgctgacagatcctacttcctgtacagcaagctgcgcgtggacagaaacagctggcagaggggaggaacctacacctgcgtggtcatgcacgaggccctgcacaaccactacatgcagaagagcacctccaagagcgccggcaagcacgagacaacacctaacaagggcagcggaactaccagcggcaccacccggctgctgagcggccacacctgcttcaccctgacaggcctgctgggcaccctggtcacaatgggcctgctcacctgatga

SEQ ID NO: 30 - DNA, 123 bp, synthetic construct
  source 1..123: mol_type = other DNA
cacgagacaacacctaacaagggcagcggaactaccagcggcaccacccggctgctgagcggccacacctgcttcaccctgacaggcctgctgggcaccctggtcacaatgggcctgctcacc

SEQ ID NO: 31 - DNA, 1812 bp, synthetic construct
  source 1..1812: mol_type = other DNA
aagctttaatacgactcactatagggaaataagagagaaaagaagagtaagaagaaatataagagccaccatggccgtgctgggcctgctgttctgcctggtgaccttcccttcctgcgtgctgagccaggtgcagctgaagcaggctggacctggactggtgcagccttcccagagcctgtccatcacctgcaccgtgtccggcttcagcctgtccaactacggagtgcactgggtgcggcagagccctggcaagggcctggagtggctgggactgatctggggaggcggccggtccgactacaacgctgccttcatctccaggctgaccatcagcaaggacaacagcaagtcccaggtgttcttcaccatgaacagcctgcaggtgaacgacaccggcatctactactgcgtgcggaacggaggcgaccctgcttggttcgcttactggggacagggcaccctggtgaccgtgtccgccgctagcacctccgcccccaaggtgtaccctctgaccagctgctgcggcgacaagagctccagcagggtgaccctgggatgcctggtgtccagctacatgcccgagcctgtgaccgtgacctggaactccggcgccctgaagagcggagtgcacaccttccctgccgtgctgcagtccagcggactgtactccctgtccagcatggtgaccgtgcctggaagcacctccggacagaccttcacctgcaacgtggctcaccctgcttccagcaccaaggtggacaaggccgtgggcgtgtccagcgactgctccaagcccaacaaccagcactgcgtgcgggagcctagcgtgttcatcttcccccctaagcccaaggacaccctgatgatcaccggaacccctgaggtgacctgcgtggtggtgaacgtgggccacgacaaccccgaggtgcagttcagctggttcgtggacgacgtggaggtgcacaccgctaggaccaagcctagggaggagcagttcaactccacctacagggtggtgagcgccctgcctatccagcaccaggactggaccggaggcaaggagttcaagtgcaaggtgaacatcaagggcctgagcgcctccatcgtgcgcatcatcagcagatccaagggccccgccagagagcctcaggtgtacgtgctggacccccctaaggaggagctgagcaagtccaccgtgtccgtgacctgcatggtcatcggcttctaccctgaggacgtggacgtggagtggcagagggacaggcagaccgagagcgaggacaagtacaggaccacccctcctcagctggacgctgacagatcctacttcctgtacagcaagctgcgcgtggacagaaacagctggcagaggggaggaacctacacctgcgtggtcatgcacgaggccctgcacaaccactacatgcagaagagcacctccaagagcgccggcaagcacgagacaacacctaacaagggcagcggaactaccagcggcaccacccggctgctgagcggccacacctgcttcaccctgacaggcctgctgggcaccctggtcacaatgggcctgctcacctgatgacacgagacaacacctaacaagggcagcggaactaccagcggcaccacccggctgctgagcggccacacctgcttcaccctgacaggcctgctgggcaccctggtcacaatgggcctgctcaccgctgccttctgcggggcttgccttctggccatgcccttcttctctcccttgcacctgtacctcttggtctttgaataaagcctgagtaggaaggcggccgc

SEQ ID NO: 32 - skipped

SEQ ID NO: 33 - DNA, 15 bp, synthetic construct
  source 1..15: mol_type = other DNA
ggaggaggaggaagc

SEQ ID NO: 34 - skipped

SEQ ID NO: 35 - DNA, 1935 bp, synthetic construct
  source 1..1935: mol_type = other DNA
aagctttaatacgactcactatagggaaataagagagaaaagaagagtaagaagaaatataagagccaccatggcctggatcagcctgatcctgtccctgctggccctgagctccggagccatcagccaggctgtggtgacccaggagagcgccctgaccacctcccctggagagaccgtgaccctgacctgccgcagctccaccggagctgtgaccacctccaactacgccaactgggtgcaggagaagcctgaccacctgttcaccggcctgatcggaggaaccaacaacagggctcctggagtgcctgccaggttctccggcagcctgatcggcgacaaggccgccctgaccatcaccggagctcagaccgaggacgaggccatctacttctgcgccctgtggtacagcaacctgtgggtgttcggaggaggaaccaagctgaccgtgctgcggagcgacgctgagccttccgtgttcctgttcaagcctagcgacgagcagctgaagaccggcaccgtgtccgtggtgtgcctggtgaacgacttctaccccaaggacatcaacgtgaagtggaaggtggacggcgtgacccagagctccagcaacttccagaacagcttcaccgaccaggactccaagaagagcacctactccctgtccagcatcctgaccctgccttccagcgagtaccagtcccacgacgcctacacctgcgaggtgagccacaagtccctgaccaccaccctggtgaagtccttcagcaagaacgagtgcggaggaggaggaagcgtgttcaccctggaggactttgtgggcgattggaggcagaccgccggctataacctggaccaggtgctggagcagggaggcgtgtcctctctgtttcagaatctgggcgtgtccgtgaccccaatccagaggatcgtgctgtctggcgagaacggcctgaagatcgacatccacgtgatcatcccttacgagggcctgtccggcgatcagatgggccagatcgagaagatcttcaaggtggtgtacccagtggacgatcaccacttcaaagtgatcctgcactatggcaccctggtcatcgacggcgtgaccccaaatatgatcgattacttcggcagaccctatgagggcatcgccgtgtttgatggcaagaagatcacagtgaccggcacactgtggaacggcaataagatcatcgacgagcggctgatcaaccccgatggcagcctgctgttcagagtgacaatcaatggagtgaccggatggaggctgtgcgagagaatcctggcctgatgaggaggaggaggaagcgtgttcaccctggaggactttgtgggcgattggaggcagaccgccggctataacctggaccaggtgctggagcagggaggcgtgtcctctctgtttcagaatctgggcgtgtccgtgaccccaatccagaggatcgtgctgtctggcgagaacggcctgaagatcgacatccacgtgatcatcccttacgagggcctgtccggcgatcagatgggccagatcgagaagatcttcaaggtggtgtacccagtggacgatcaccacttcaaagtgatcctgcactatggcaccctggtcatcgacggcgtgaccccaaatatgatcgattacttcggcagaccctatgagggcatcgccgtgtttgatggcaagaagatcacagtgaccggcacactgtggaacggcaataagatcatcgacgagcggctgatcaaccccgatggcagcctgctgttcagagtgacaatcaatggagtgaccggatggaggctgtgcgagagaatcctggccgctgccttctgcggggcttgccttctggccatgcccttcttctctcccttgcacctgtacctcttggtctttgaataaagcctgagtaggaaggcggccgc

SEQ ID NO: 36 - DNA, 70 bp, synthetic construct; identical to SEQ ID NO: 1
  source 1..70: mol_type = other DNA

SEQ ID NO: 37 - DNA, 241 bp, synthetic construct
  source 1..241: mol_type = other DNA
aagctttaatacgactcactatagggaaataagagagaaaagaagagtaagaagaaatataagagccaccaagctttaatacgactcactatagggaaataagagagaaaagaagagtaagaagaaatataagagccaccgctgccttctgcggggcttgccttctggccatgcccttcttctctcccttgcacctgtacctcttggtctttgaataaagcctgagtaggaaggcggccgc

SEQ ID NO: 38 - DNA, 729 bp, synthetic construct
  source 1..729: mol_type = other DNA
atggacagccaggcccaggtgctgatgctgctgctgctgtgggtgtccggaacctgcggcgacatcgtgatgtcccagagccctagctccctggccgtgagcgtgggagagaaggtgaccatgagctgcaagagctcccagtccctgctgtacagctccaaccagaagaactacctggcctggtaccagcagaagcccggccagagccctaagctgctgatctactgggctagcaccagggagtccggagtgcctgacaggttcaccggatccggaagcggaaccgacttcaccctgactatcagctccgtgaaggccgaggacctggccgtgtactactgccagcagtactacaactaccctttcaccttcggcggcggcaccaacctggagatcaagcggagcgacgctgagccttccgtgttcctgttcaagcctagcgacgagcagctgaagaccggcaccgtgtccgtggtgtgcctggtgaacgacttctaccccaaggacatcaacgtgaagtggaaggtggacggcgtgacccagagctccagcaacttccagaacagcttcaccgaccaggactccaagaagagcacctactccctgtccagcatcctgaccctgccttccagcgagtaccagtcccacgacgcctacacctgcgaggtgagccacaagtccctgaccaccaccctggtgaagtccttcagcaagaacgagtgctgatga

SEQ ID NO: 39 - DNA, 900 bp, synthetic construct
  source 1..900: mol_type = other DNA
aagctttaatacgactcactatagggaaataagagagaaaagaagagtaagaagaaatataagagccaccatggacagccaggcccaggtgctgatgctgctgctgctgtgggtgtccggaacctgcggcgacatcgtgatgtcccagagccctagctccctggccgtgagcgtgggagagaaggtgaccatgagctgcaagagctcccagtccctgctgtacagctccaaccagaagaactacctggcctggtaccagcagaagcccggccagagccctaagctgctgatctactgggctagcaccagggagtccggagtgcctgacaggttcaccggatccggaagcggaaccgacttcaccctgactatcagctccgtgaaggccgaggacctggccgtgtactactgccagcagtactacaactaccctttcaccttcggcggcggcaccaacctggagatcaagcggagcgacgctgagccttccgtgttcctgttcaagcctagcgacgagcagctgaagaccggcaccgtgtccgtggtgtgcctggtgaacgacttctaccccaaggacatcaacgtgaagtggaaggtggacggcgtgacccagagctccagcaacttccagaacagcttcaccgaccaggactccaagaagagcacctactccctgtccagcatcctgaccctgccttccagcgagtaccagtcccacgacgcctacacctgcgaggtgagccacaagtccctgaccaccaccctggtgaagtccttcagcaagaacgagtgctgatgagctgccttctgcggggcttgccttctggccatgcccttcttctctcccttgcacctgtacctcttggtctttgaataaagcctgagtaggaaggcggccgc

SEQ ID NO: 40 - DNA, 1512 bp, synthetic construct
  source 1..1512: mol_type = other DNA
atggccgtgctggtgctgctgctgtgcctggtgaccttccctagctgcgtgctgtcccaggtgcagctgaagcagagcggacctggactggtgcagcctagccagaacctgtccatcacctgcaccgtgagcggcttctccctgaccacctacggagtgcactgggtgcgccagagccctggcaagggcctggactggctgggcgtgatctggggaggaggatccaccgacttcaacgctgccttcatgagcagactgtccatcaccaaggacaactccaagagccaggtgttcttcaagatgaacagcctgcaggccgacgacaccgccatctactactgcgccaagagcctgctgagctccatggactactggggacagggcacctccgtgaccgtgagctccgcctccaccagcgcccctaaggtgtaccctctgacctcctgctgcggcgacaagagctccagcagggtgaccctgggatgcctggtgtccagctacatgcccgagcctgtgaccgtgacctggaacagcggcgccctgaagtccggagtgcacaccttccctgccgtgctgcagtccagcggactgtacagcctgtccagcatggtgaccgtgcctggatccaccagcggacagaccttcacctgcaacgtggctcaccctgcttccagcaccaaggtggacaaggccgtgggcgtgtccagcgactgcagcaagcccaacaaccagcactgcgtgagagagccttccgtgttcatcttcccccctaagcccaaggacaccctgatgatcaccggaacccctgaggtgacctgcgtggtggtgaacgtgggccacgacaaccccgaggtgcagttctcctggttcgtggacgacgtggaggtgcacaccgctaggaccaagcctagggaggagcagttcaacagcacctacagggtggtgtccgccctgcctatccagcaccaggactggaccggaggcaaggagttcaagtgcaaggtgaacatcaagggcctgtccgccagcatcgtgcgcatcatctccagaagcaagggacctgctagggagcctcaggtgtacgtgctggacccccctaaggaggagctgtccaagagcaccgtgagcgtgacctgcatggtcatcggcttctaccctgaggacgtggacgtggagtggcagagggacaggcagaccgagtccgaggacaagtacaggaccacccctcctcagctggacgctgacaggagctacttcctgtactccaagctgcgcgtggacagaaacagctggcagaggggaggaacctacacctgcgtggtcatgcacgaggccctgcacaaccactacatgcagaagtccaccagcaagtccgccggcaagcacgagacaacacctaacaagggcagcggaactaccagcggcaccacccggctgctgagcggccacacctgcttcaccctgacaggcctgctgggcaccctggtcacaatgggcctgctcacctgatga

SEQ ID NO: 41 - DNA, 1806 bp, synthetic construct
  source 1..1806: mol_type = other DNA
aagctttaatacgactcactatagggaaataagagagaaaagaagagtaagaagaaatataagagccaccatggccgtgctggtgctgctgctgtgcctggtgaccttccctagctgcgtgctgtcccaggtgcagctgaagcagagcggacctggactggtgcagcctagccagaacctgtccatcacctgcaccgtgagcggcttctccctgaccacctacggagtgcactgggtgcgccagagccctggcaagggcctggactggctgggcgtgatctggggaggaggatccaccgacttcaacgctgccttcatgagcagactgtccatcaccaaggacaactccaagagccaggtgttcttcaagatgaacagcctgcaggccgacgacaccgccatctactactgcgccaagagcctgctgagctccatggactactggggacagggcacctccgtgaccgtgagctccgcctccaccagcgcccctaaggtgtaccctctgacctcctgctgcggcgacaagagctccagcagggtgaccctgggatgcctggtgtccagctacatgcccgagcctgtgaccgtgacctggaacagcggcgccctgaagtccggagtgcacaccttccctgccgtgctgcagtccagcggactgtacagcctgtccagcatggtgaccgtgcctggatccaccagcggacagaccttcacctgcaacgtggctcaccctgcttccagcaccaaggtggacaaggccgtgggcgtgtccagcgactgcagcaagcccaacaaccagcactgcgtgagagagccttccgtgttcatcttcccccctaagcccaaggacaccctgatgatcaccggaacccctgaggtgacctgcgtggtggtgaacgtgggccacgacaaccccgaggtgcagttctcctggttcgtggacgacgtggaggtgcacaccgctaggaccaagcctagggaggagcagttcaacagcacctacagggtggtgtccgccctgcctatccagcaccaggactggaccggaggcaaggagttcaagtgcaaggtgaacatcaagggcctgtccgccagcatcgtgcgcatcatctccagaagcaagggacctgctagggagcctcaggtgtacgtgctggacccccctaaggaggagctgtccaagagcaccgtgagcgtgacctgcatggtcatcggcttctaccctgaggacgtggacgtggagtggcagagggacaggcagaccgagtccgaggacaagtacaggaccacccctcctcagctggacgctgacaggagctacttcctgtactccaagctgcgcgtggacagaaacagctggcagaggggaggaacctacacctgcgtggtcatgcacgaggccctgcacaaccactacatgcagaagtccaccagcaagtccgccggcaagcacgagacaacacctaacaagggcagcggaactaccagcggcaccacccggctgctgagcggccacacctgcttcaccctgacaggcctgctgggcaccctggtcacaatgggcctgctcacctgatgacacgagacaacacctaacaagggcagcggaactaccagcggcaccacccggctgctgagcggccacacctgcttcaccctgacaggcctgctgggcaccctggtcacaatgggcctgctcaccgctgccttctgcggggcttgccttctggccatgcccttcttctctcccttgcacctgtacctcttggtctttgaataaagcctgagtaggaaggcggccgc

SEQ ID NO: 42 - DNA, 1254 bp, synthetic construct
  source 1..1254: mol_type = other DNA
atggacagccaggcccaggtgctgatgctgctgctgctgtgggtgtccggaacctgcggcgacatcgtgatgtcccagagccctagctccctggccgtgagcgtgggagagaaggtgaccatgagctgcaagagctcccagtccctgctgtacagctccaaccagaagaactacctggcctggtaccagcagaagcccggccagagccctaagctgctgatctactgggctagcaccagggagtccggagtgcctgacaggttcaccggatccggaagcggaaccgacttcaccctgactatcagctccgtgaaggccgaggacctggccgtgtactactgccagcagtactacaactaccctttcaccttcggcggcggcaccaacctggagatcaagcggagcgacgctgagccttccgtgttcctgttcaagcctagcgacgagcagctgaagaccggcaccgtgtccgtggtgtgcctggtgaacgacttctaccccaaggacatcaacgtgaagtggaaggtggacggcgtgacccagagctccagcaacttccagaacagcttcaccgaccaggactccaagaagagcacctactccctgtccagcatcctgaccctgccttccagcgagtaccagtcccacgacgcctacacctgcgaggtgagccacaagtccctgaccaccaccctggtgaagtccttcagcaagaacgagtgcggaggaggaggaagcgtgttcaccctggaggactttgtgggcgattggaggcagaccgccggctataacctggaccaggtgctggagcagggaggcgtgtcctctctgtttcagaatctgggcgtgtccgtgaccccaatccagaggatcgtgctgtctggcgagaacggcctgaagatcgacatccacgtgatcatcccttacgagggcctgtccggcgatcagatgggccagatcgagaagatcttcaaggtggtgtacccagtggacgatcaccacttcaaagtgatcctgcactatggcaccctggtcatcgacggcgtgaccccaaatatgatcgattacttcggcagaccctatgagggcatcgccgtgtttgatggcaagaagatcacagtgaccggcacactgtggaacggcaataagatcatcgacgagcggctgatcaaccccgatggcagcctgctgttcagagtgacaatcaatggagtgaccggatggaggctgtgcgagagaatcctggcctgatga

SEQ ID NO: 43 - DNA, 510 bp, synthetic construct
  source 1..510: mol_type = other DNA
gtgttcaccctggaggactttgtgggcgattggaggcagaccgccggctataacctggaccaggtgctggagcagggaggcgtgtcctctctgtttcagaatctgggcgtgtccgtgaccccaatccagaggatcgtgctgtctggcgagaacggcctgaagatcgacatccacgtgatcatcccttacgagggcctgtccggcgatcagatgggccagatcgagaagatcttcaaggtggtgtacccagtggacgatcaccacttcaaagtgatcctgcactatggcaccctggtcatcgacggcgtgaccccaaatatgatcgattacttcggcagaccctatgagggcatcgccgtgtttgatggcaagaagatcacagtgaccggcacactgtggaacggcaataagatcatcgacgagcggctgatcaaccccgatggcagcctgctgttcagagtgacaatcaatggagtgaccggatggaggctgtgcgagagaatcctggcc

SEQ ID NO: 44 - DNA, 1950 bp, synthetic construct
  source 1..1950: mol_type = other DNA
aagctttaatacgactcactatagggaaataagagagaaaagaagagtaagaagaaatataagagccaccatggacagccaggcccaggtgctgatgctgctgctgctgtgggtgtccggaacctgcggcgacatcgtgatgtcccagagccctagctccctggccgtgagcgtgggagagaaggtgaccatgagctgcaagagctcccagtccctgctgtacagctccaaccagaagaactacctggcctggtaccagcagaagcccggccagagccctaagctgctgatctactgggctagcaccagggagtccggagtgcctgacaggttcaccggatccggaagcggaaccgacttcaccctgactatcagctccgtgaaggccgaggacctggccgtgtactactgccagcagtactacaactaccctttcaccttcggcggcggcaccaacctggagatcaagcggagcgacgctgagccttccgtgttcctgttcaagcctagcgacgagcagctgaagaccggcaccgtgtccgtggtgtgcctggtgaacgacttctaccccaaggacatcaacgtgaagtggaaggtggacggcgtgacccagagctccagcaacttccagaacagcttcaccgaccaggactccaagaagagcacctactccctgtccagcatcctgaccctgccttccagcgagtaccagtcccacgacgcctacacctgcgaggtgagccacaagtccctgaccaccaccctggtgaagtccttcagcaagaacgagtgcggaggaggaggaagcgtgttcaccctggaggactttgtgggcgattggaggcagaccgccggctataacctggaccaggtgctggagcagggaggcgtgtcctctctgtttcagaatctgggcgtgtccgtgaccccaatccagaggatcgtgctgtctggcgagaacggcctgaagatcgacatccacgtgatcatcccttacgagggcctgtccggcgatcagatgggccagatcgagaagatcttcaaggtggtgtacccagtggacgatcaccacttcaaagtgatcctgcactatggcaccctggtcatcgacggcgtgaccccaaatatgatcgattacttcggcagaccctatgagggcatcgccgtgtttgatggcaagaagatcacagtgaccggcacactgtggaacggcaataagatcatcgacgagcggctgatcaaccccgatggcagcctgctgttcagagtgacaatcaatggagtgaccggatggaggctgtgcgagagaatcctggcctgatgaggaggaggaggaagcgtgttcaccctggaggactttgtgggcgattggaggcagaccgccggctataacctggaccaggtgctggagcagggaggcgtgtcctctctgtttcagaatctgggcgtgtccgtgaccccaatccagaggatcgtgctgtctggcgagaacggcctgaagatcgacatccacgtgatcatcccttacgagggcctgtccggcgatcagatgggccagatcgagaagatcttcaaggtggtgtacccagtggacgatcaccacttcaaagtgatcctgcactatggcaccctggtcatcgacggcgtgaccccaaatatgatcgattacttcggcagaccctatgagggcatcgccgtgtttgatggcaagaagatcacagtgaccggcacactgtggaacggcaataagatcatcgacgagcggctgatcaaccccgatggcagcctgctgttcagagtgacaatcaatggagtgaccggatggaggctgtgcgagagaatcctggccgctgccttctgcggggcttgccttctggccatgcccttcttctctcccttgcacctgtacctcttggtctttgaataaagcctgagtaggaaggcggccgc